SEQUENCE LISTING (42 sequences)

SEQ ID NO: 1 - protein, 10 aa, synthetic construct
SAWSHPQFEK

SEQ ID NO: 2 - protein, 8 aa, synthetic construct
DYKDDDDK

SEQ ID NO: 3 - protein, 88 aa, synthetic construct
MWTLVSWVALTAGLVAGSAWSHPQFEKGGGSGGGSGGSAWSHPQFEKGASDYKDDDDKTRCPDGQFCPVACCLDPGGASYSCCRPLLD

SEQ ID NO: 4 - protein, 115 aa, synthetic construct
MWTLVSWVALTAGLVAGSAWSHPQFEKGGGSGGGSGGSAWSHPQFEKGASDYKDDDDKGGPCQVDAHCSAGHSCIFTVSGTSSCCPFPEAVACGDGHHCCPRGFHCSADGRSCFQ

SEQ ID NO: 5 - protein, 116 aa, synthetic construct
MWTLVSWVALTAGLVAGSAWSHPQFEKGGGSGGGSGGSAWSHPQFEKGASDYKDDDDKAIQCPDSQFECPDFSTCCVMVDGSWGCCPMPQASCCEDRVHCCPHGAFCDLVHTRCIT

SEQ ID NO: 6 - protein, 116 aa, synthetic construct
MWTLVSWVALTAGLVAGSAWSHPQFEKGGGSGGGSGGSAWSHPQFEKGASDYKDDDDKSVMCPDARSRCPDGSTCCELPSGKYGCCPMPNATCCSDHLHCCPQDTVCDLIQSKCLS

SEQ ID NO: 7 - protein, 115 aa, synthetic construct
MWTLVSWVALTAGLVAGSAWSHPQFEKGGGSGGGSGGSAWSHPQFEKGASDYKDDDDKDVKCDMEVSCPDGYTCCRLQSGAWGCCPFTQAVCCEDHIHCCPAGFTCDTQKGTCEQ

SEQ ID NO: 8 - protein, 114 aa, synthetic construct
MWTLVSWVALTAGLVAGSAWSHPQFEKGGGSGGGSGGSAWSHPQFEKGASDYKDDDDKDVPCDNVSSCPSSDTCCQLTSGEWGCCPIPEAVCCSDHQHCCPQGYTCVAEGQCQR

SEQ ID NO: 9 - protein, 115 aa, synthetic construct
MWTLVSWVALTAGLVAGSAWSHPQFEKGGGSGGGSGGSAWSHPQFEKGASDYKDDDDKDIGCDQHTSCPVGQTCCPSLGGSWACCQLPHAVCCEDRQHCCPAGYTCNVKARSCEK

SEQ ID NO: 10 - protein, 115 aa, synthetic construct
MWTLVSWVALTAGLVAGSAWSHPQFEKGGGSGGGSGGSAWSHPQFEKGASDYKDDDDKDVECGEGHFCHDNQTCCRDNRQGWACCPYRQGVCCADRRHCCPAGFRCAARGTKCLR

SEQ ID NO: 11 - protein, 146 aa, synthetic construct
MWTLVSWVALTAGLVAGSAWSHPQFEKGGGSGGGSGGSAWSHPQFEKGASDYKDDDDKAIQCPDSQFECPDFSTCCVMVDGSWGCCPMPQASCCEDRVHCCPHGAFCDLVHTRCITPTGTHPLAKKLPAQRTNRAVALSSALRQLL

SEQ ID NO: 12 - protein, 98 aa, synthetic construct
MWTLVSWVALTAGLVAGSAWSHPQFEKGGGSGGGSGGSAWSHPQFEKGASDYKDDDDKTRCPDGQFCPVACCLDPGGASYSCCRPLLDKWPTTLSRHL

SEQ ID NO: 13 - protein, 123 aa, synthetic construct
MWTLVSWVALTAGLVAGSAWSHPQFEKGGGSGGGSGGSAWSHPQFEKGASDYKDDDDKGGPCQVDAHCSAGHSCIFTVSGTSSCCPFPEAVACGDGHHCCPRGFHCSADGRSCFQRSGNNSVG

SEQ ID NO: 14 - protein, 140 aa, synthetic construct
MWTLVSWVALTAGLVAGSAWSHPQFEKGGGSGGGSGGSAWSHPQFEKGASDYKDDDDKAIQCPDSQFECPDFSTCCVMVDGSWGCCPMPQASCCEDRVHCCPHGAFCDLVHTRCITPTGTHPLAKKLPAQRTNRAVALSS

SEQ ID NO: 15 - protein, 134 aa, synthetic construct
MWTLVSWVALTAGLVAGSAWSHPQFEKGGGSGGGSGGSAWSHPQFEKGASDYKDDDDKSVMCPDARSRCPDGSTCCELPSGKYGCCPMPNATCCSDHLHCCPQDTVCDLIQSKCLSKENATTDLLTKLPAHTVG

SEQ ID NO: 16 - protein, 140 aa, synthetic construct
MWTLVSWVALTAGLVAGSAWSHPQFEKGGGSGGGSGGSAWSHPQFEKGASDYKDDDDKDVKCDMEVSCPDGYTCCRLQSGAWGCCPFTQAVCCEDHIHCCPAGFTCDTQKGTCEQGPHQVPWMEKAPAHLSLPDPQALKR

SEQ ID NO: 17 - protein, 136 aa, synthetic construct
MWTLVSWVALTAGLVAGSAWSHPQFEKGGGSGGGSGGSAWSHPQFEKGASDYKDDDDKDVPCDNVSSCPSSDTCCQLTSGEWGCCPIPEAVCCSDHQHCCPQGYTCVAEGQCQRGSEIVAGLEKMPARRASLSHPR

SEQ ID NO: 18 - protein, 135 aa, synthetic construct
MWTLVSWVALTAGLVAGSAWSHPQFEKGGGSGGGSGGSAWSHPQFEKGASDYKDDDDKDIGCDQHTSCPVGQTCCPSLGGSWACCQLPHAVCCEDRQHCCPAGYTCNVKARSCEKEVVSAQPATFLARSPHVGVK

SEQ ID NO: 19 - protein, 134 aa, synthetic construct
MWTLVSWVALTAGLVAGSAWSHPQFEKGGGSGGGSGGSAWSHPQFEKGASDYKDDDDKDVECGEGHFCHDNQTCCRDNRQGWACCPYRQGVCCADRRHCCPAGFRCAARGTKCLRREAPRWDAPLRDPALRQLL

SEQ ID NO: 20 - protein, 57 aa, synthetic construct
GGPCQVDAHCSAGHSCIFTVSGTSSCCPFPEAVACGDGHHCCPRGFHCSADGRSCFQ

SEQ ID NO: 21 - protein, 58 aa, synthetic construct
AIQCPDSQFECPDFSTCCVMVDGSWGCCPMPQASCCEDRVHCCPHGAFCDLVHTRCIT

SEQ ID NO: 22 - protein, 42 aa, synthetic construct
XCXDXXTCCXYXXXXXGCCPMPXAXCCXDXXHCCPXXXXCDX

SEQ ID NO: 23 - protein, 57 aa, synthetic construct
VMCPDARSRCPDGSTCCELPSGKYGCCPMPNATCCSDHLHCCPQDTVCDLIQSKCLS

SEQ ID NO: 24 - protein, 54 aa, synthetic construct
CDMEVSCPDGYTCCRLQSGAWGCCPFTQAVCCEDHIHCCPAGFTCDTQKGTCEQ

SEQ ID NO: 25 - protein, 56 aa, synthetic construct
DVPCDNVSSCPSSDTCCQLTSGEWGCCPIPEAVCCSDHQHCCPQGYTCVAEGQCQR

SEQ ID NO: 26 - protein, 57 aa, synthetic construct
DIGCDQHTSCPVGQTCCPSLGGSWACCQLPHAVCCEDRQHCCPAGYTCNVKARSCEK

SEQ ID NO: 27 - protein, 57 aa, synthetic construct
DVECGEGHFCHDNQTCCRDNRQGWACCPYRQGVCCADRRHCCPAGFRCAARGTKCLR

SEQ ID NO: 28 - protein, 30 aa, synthetic construct
TRCPDGQFCPVACCLDPGGASYSCCRPLLD

SEQ ID NO: 29 - protein, 17 aa, synthetic construct
MWTLVSWVALTAGLVAG

SEQ ID NO: 30 - protein, 40 aa, synthetic construct
AWSHPQFEKGGGSGGGSGGSAWSHPQFEKGASDYKDDDDK

SEQ ID NO: 31 - protein, 42 aa, synthetic construct
ECPDFSTCCVMVDGSWGCCPMPQASCCEDRVHCCPHGAFCDL

SEQ ID NO: 32 - protein, 42 aa, synthetic construct
QCPDDSTCCELPTGKYGCCPMPNAICCSDHLHCCPQDTVCDL

SEQ ID NO: 33 - protein, 42 aa, synthetic construct
ECSDDETCCKLGDNTWGCCPMPNAVCCDDRSHCCPTGTTCDP

SEQ ID NO: 34 - protein, 24 aa, synthetic construct
PTGTHPLAKKLPAQRTNRAVALSS

SEQ ID NO: 35 - protein, 6 aa, synthetic construct
ALRQLL

SEQ ID NO: 36 - protein, 6 aa, synthetic construct
GGGGGG

SEQ ID NO: 37 - protein, 5 aa, synthetic construct
GGGGT

SEQ ID NO: 38 - protein, 6 aa, synthetic construct
GGGPPP

SEQ ID NO: 39 - protein, 7 aa, synthetic construct
GGGAPPP

SEQ ID NO: 40 - protein, 6 aa, synthetic construct
AAGCTT

SEQ ID NO: 41 - protein, 6 aa, synthetic construct
GCCACC

SEQ ID NO: 42 - protein, 9 aa, synthetic construct
TGACTCGAG

(19) United States
(12) Patent Application Publication
(10) Pub. No.: US 2024/0002459 A1
(43) Pub. Date: Jan. 4, 2024

(54) Granulin Compositions and Uses Related Thereto

(71) Applicant: Emory University, Atlanta, GA (US)

(72) Inventors: Thomas Kukar, Atlanta, GA (US); Christopher Holler, Arlington, MA (US); Georgia Taylor, Atlanta, GA (US)

(21) Appl. No.: 18/176,859

(22) Filed: Mar. 1, 2023

Related U.S. Application Data
(63) Continuation-in-part of application No. 16/317,731, filed on Jan. 14, 2019, now abandoned.
(60) Provisional application No. 62/362,367, filed on Jul. 14, 2016.

(51) Int. Cl. C07K 14/475; C12N 15/86; A61P 25/28; A61K 38/18

(52) U.S. Cl. CPC C07K 14/475; C12N 15/86; A61P 25/28; A61K 38/18

(57) ABSTRACT

This disclosure relates to purified and recombinant granulins, fusions, or variants and vectors encoding the same. In certain embodiments, this disclosure relates to uses of these granulins or vectors, alone or in combination, in the treatment or prevention of diseases or conditions associated with lysosomal defects such as frontotemporal dementia and Alzheimer's disease.

### CROSS-REFERENCE TO RELATED APPLICATIONS

This application is a continuation in part of U.S. application Ser. No. 16/317,731 filed Jan. 14, 2019, which is the National Stage of International Application No. PCT/US2017/041879 filed Jul. 13, 2017, which claims the benefit of U.S. Provisional Application No. 62/362,367 filed Jul. 14, 2016. The entirety of each of these applications are hereby incorporated by reference for all purposes.

### STATEMENT REGARDING FEDERALLY SPONSORED RESEARCH OR DEVELOPMENT

This invention was made with government support under NS093362 and AG032362 awarded by the National Institutes of Health. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML, format and is hereby incorporated by reference into the specification. The name of the XML file containing the Sequence Listing is 16127USCIP.xml. The XML file is 39 KB, was created on Feb. 28, 2023, and is being submitted electronically via the USPTO patent electronic filing system.

### BACKGROUND

Shoyab et al. report epithelins, also called granulins (GRNs), are low molecular mass (approximately 6 kDa) single-chain proteins containing cysteine rich sequences. Proc Natl Acad Sci USA. 1990, 87(20):7912-6. GRNs are generated by the proteolytic cleavage of progranulin (PGRN). Within PGRN, each GRN is joined by short linkers sequences that can be cleaved to release the mature GRN proteins. The GRNs were originally named using letters (A-G plus paragranulin). The consensus nomenclature (UniProtKB: P28799) refers to each GRN numerically according to their position within PGRN starting at the amino-terminus as follows: Para-GRN (paragranulin), GRN-1 (G), GRN-2 (F), GRN-3 (B), GRN-4 (A), GRN-5 (C), GRN-6 (D), GRN-7 (E).

Mutations in the GRN gene cause neuronal ceroid lipofuscinosis (NCL) or frontotemporal dementia (FTD). NCL and FTD are characterized by neurodegeneration and lysosome dysfunction indicating PGRN is important for lysosome homeostasis in the brain. GRN mutations cause FTD through haploinsufficiency or loss of function of PGRN. See Meeter et al. Dement Geriatr Cogn Disord Extra, 2016, 6:330-340 and Pottier et al. J Neurochem, 2016, 138 Suppl 1:32-53. Although PGRN can traffic to the lysosome, its function within the lysosome is unknown.

Salazar et al. report granulins exacerbate TDP-43 toxicity and increase TDP-43 Levels. J Neurosc, 2015 35:9315-9328.

Hu et al. report sortilin-mediated endocytosis determines levels of the frontotemporal dementia protein, progranulin. Neuron, 2010, 68:654-667.

Zhou et al. report prosaposin facilitates sortilin-independent lysosomal trafficking of progranulin. J Cell Biol, 2015, 210:991-1002.

References cited herein are not an admission of prior art.

### SUMMARY

This disclosure relates to purified and recombinant granulins, fusions, or variants and vectors encoding the same. In certain embodiments, this disclosure relates to uses of these granulins or vectors, alone or in combination, in the treatment or prevention of diseases or conditions associated with lysosomal defects such as frontotemporal dementia and Alzheimer's disease.

In certain embodiments, the granulins comprise or consist of any of the sequences disclosed herein, variants, or derivatives thereof. In certain embodiments, the variants have one, two, three, or more amino acid substitutions, insertions, or deletions. In certain embodiments, the granulins are produced synthetically or recombinantly. In certain embodiments, the granulins have at least one non-naturally occurring amino acid substitution, addition, or deletion. In certain embodiments, the amino acid substitutions are conserved substitutions.

In certain embodiments, the disclosure contemplates fusion proteins comprising a granulin or variant disclosed herein. In certain embodiments, the disclosure contemplates granulins disclosed herein having at least one molecular modification, e.g., such that the granulin contains a non-naturally amino acid. In certain embodiments, the disclosure contemplates a non-naturally occurring derivative of a granulin having any of the sequences disclosed herein variants, or derivatives thereof. In certain embodiments, the disclosure contemplates a derivative in the form of a prodrug. In certain embodiments, the disclosure contemplates a derivative wherein an amino, carboxyl, hydroxyl, or thiol group in a granulin peptide disclosed herein is substituted, e.g., wherein an amino acid side chain, the C- or N-terminus, is substituted. In certain embodiments, the disclosure contemplates peptides disclosed herein having a label, e.g., fluorescent or radioactive label.

In certain embodiments, the disclosure contemplates a granulin having at least 50%, 60%, 70%, 80%, 90%, 95% sequence identity or similarity to any of the sequences disclosed herein, and contains at least one substitution and/or modification such that the entire peptide is not naturally occurring, e.g., one or more amino acids have been changed relative to the natural sequence.

In certain embodiments, the variants are any of the sequences disclosed herein with greater than 70, 80, 90, 95, or 98% sequence identity or similarity. In certain embodiments, the recombinant granulins or variants comprise a signal sequence, a granulin sequence or variant, and optionally a tag sequence or variant. In certain embodiments, the recombinant granulins or variants comprise a signal sequence or variant, a paragranulin sequence or variant, a granulin sequence or variant, and optionally a tag sequence or variant.

In certain embodiments, the recombinant granulins comprises or consist of granulin-1 or a variant with greater than 70, 80, 90, 95, or 98% sequence identity or similarity to GGPCQVDAHCSAGHSCIFTVSGTSSCCPFPEAVACGDGHHCCPRGFHCSADGRSCFQ, (SEQ ID NO: 20).

In certain embodiments, the recombinant granulins comprises or consist of granulin-2 or a variant with greater than 70, 80, 90, 95, or 98% sequence identity or similarity to AIQCPDSQFECPDFSTCCVMVDGSWGCCPMPQASCCEDRVHCCPHGAFCDLVHTRCIT, (SEQ ID NO: 21). In certain embodiments, the recombinant granulin-2 comprises the consensus sequence Z¹CX¹DX²X³TCCX⁴Z²X⁵X⁶X⁷X⁸Z³GCCPMPZ⁴AX⁹CCZ⁵DZ⁶X¹⁰HCCPX¹¹X¹²X¹³X¹⁴CDZ⁷ (SEQ ID NO: 22), wherein each X is, individually and independently, any amino acid and each Z is, individually and independently, any amino acid or a conserved or similar amino acid compared to the corresponding amino acid when aligned with SEQ ID NO: 21.

In certain embodiments, the recombinant granulins comprises or consist of granulin-3 or a variant with greater than 70, 80, 90, 95, or 98% sequence identity or similarity to, VMCPDARSRCPDGSTCCELPSGKYGCCPMPNATCCSDHLHCCPQDTVCDLIQSKCLS (SEQ ID NO: 23).

In certain embodiments, the recombinant granulins comprises or consist of granulin-4 or a variant with greater than 70, 80, 90, 95, or 98% sequence identity or similarity to, CDMEVSCPDGYTCCRLQSGAWGCCPFTQAVCCEDHIHCCPAGFTCDTQKGTCEQ (SEQ ID NO: 24).

In certain embodiments, the recombinant granulins comprises or consist of granulin-5 or a variant with greater than 70, 80, 90, 95, or 98% sequence identity or similarity to, DVPCDNVSSCPSSDTCCQLTSGEWGCCPIPEAVCCSDHQHCCPQGYTCVAEGQCQR (SEQ ID NO: 25).

In certain embodiments, the recombinant granulins comprises or consist of granulin-6 or a variant with greater than 70, 80, 90, 95, or 98% sequence identity or similarity to, DIGCDQHTSCPVGQTCCPSLGGSWACCQLPHAVCCEDRQHCCPAGYTCNVKARSCEK (SEQ ID NO: 26).

In certain embodiments, the recombinant granulins comprises or consist of granulin-7 or a variant with greater than 70, 80, 90, 95, or 98% sequence identity or similarity to, DVECGEGHFCHDNQTCCRDNRQGWACCPYRQGVCCADRRHCCPAGFRCAARGTKCL R (SEQ ID NO: 27).

In certain embodiments, the recombinant granulins comprises or consist of paragranulin or a variant with greater than 70, 80, 90, 95, or 98% sequence identity or similarity to, TRCPDGQFCPVACCLDPGGASYSCCRPLLD (SEQ ID NO: 28).

In certain embodiments, recombinant granulins comprise or consist of any granulins sequence disclosed herein and an N-terminal human signal sequence or a variant with greater than 70, 80, 90, 95, or 98% sequence identity or similarity to MWTLVSWVALTAGLVAG (SEQ ID NO: 29).

In certain embodiments, recombinant granulins comprise or consist of any granulins sequence disclosed herein an N-terminal human signal sequence, and a tag sequence or a variant with greater than 70, 80, 90, 95, or 98% sequence identity or similarity to AWSHPQFEKGGGSGGGSGGSAWSHPQFEKGASDYKDDDDK (SEQ ID NO: 30).

In certain embodiments, the disclosure relates to nucleic acids encoding recombinant granulins having sequences disclosed herein or variants thereof. In certain embodiments, the nucleic acid is in a vector that is in operable combination with a promotor sequence. In certain embodiments, this disclosure contemplates vectors encoding recombinant granulins having sequences disclosed herein or variants thereof. In certain embodiments, the disclosure contemplates expression systems and cells comprising said vectors. In certain embodiments, the disclosure relates to a vector comprising the nucleic acid encoding a granulin or variant disclosed herein and a heterologous nucleic acid sequence.

In certain embodiments, the disclosure relates to a nucleic acid encoding a granulin disclosed herein wherein the nucleotide sequence has been changed to contain at least one non-naturally occurring substitution and/or modification relative to the naturally occurring sequence, e.g., one or more nucleotides have been changed relative to the natural sequence. In certain embodiments, the disclosure relates to a nucleic acid encoding a polypeptide disclosed herein further comprising a label.

In certain embodiments, the disclosure relates to pharmaceutical compositions comprising a granulin, fusion, derivative, or variant disclosed herein, or vector encoding the same and a pharmaceutically acceptable excipient. In certain embodiments, the disclosure relates to a medicament for use in managing, treating, or preventing conditions or diseases associated with lysosomal dysregulation comprising an effective amount of a granulin, fusion, derivative, or variant disclosed herein, or vector encoding the same. In certain embodiments, the pharmaceutical composition is in the form of a sterilized pH buffered aqueous salt solution. In certain embodiments, the pharmaceutical composition is in the form of a capsule, tablets, pill, powder, granule, or gel. In certain embodiments, the pharmaceutical compositions are in solid form surrounded by an enteric coating. In certain embodiments, the pharmaceutically acceptable excipient is a solubilizing agent.

In certain embodiments, the disclosure relates to methods of managing, treating, or preventing conditions or diseases associated with lysosomal dysregulation comprising administering an effective amount of a recombinant granulin or variant disclosed herein, or vector encoding the same, to a subject in need thereof. In certain embodiments, the condition or disease associated with lysosomal dysregulation is frontotemporal dementia, mild cognitive impairment, frontotemporal lobar degeneration, language impairment, progressive impairment to produce speech, deterioration of understanding words or recognizing objects, deficits in executive functioning, extrapyramidal movement disorder, motor symptoms, amyotrophic lateral sclerosis, Alzheimer's disease, Parkinson's disease, Huntington's disease (HD), and prion disease. In certain embodiments, the subject is at risk or, exhibiting symptoms of, or diagnosed with the disease or condition. In certain embodiments, administrating is by temporal vein injection (intravenous) injection, injecting directly into cerebral lateral ventricles (intracerebroventricular), or by direct injection, implantation or exposure into the brain through pressure driven infusion (convection-enhanced delivery).

### BRIEF DESCRIPTION OF THE DRAWINGS

FIG. 1A illustrates the expression of recombinant human GRNs and identification of antibodies to detect GRNs. Schematic of human GRN expression constructs. Human GRN sequences, with and without adjacent C-terminal linker regions, were synthesized to include the N-terminal PGRN signal protein (SP), followed by twin-Strep (SAWSHPQFEK)(SEQ ID NO: 1) tags and a single FLAG (DYKDDDDK)(SEQ ID NO: 2) tag. Individual GRNs are referred to by their numerical sequential designation (i.e. GRN-1, GRN-2, etc.), which correspond to their original alphabetical designation (i.e. GRN-G, GRN-F, etc.).

FIG. 1B shows data where HEK293T cells were transfected with the human GRN constructs and 48 hrs later whole-cell lysates were analyzed by immunoblot for protein expression.

FIG. 1C shows data for conditioned media analyzed by immunoblot for protein expression.

FIG. 1D shows data where GRN-expressing HEK293T cell lysates were analyzed by immunoblot to identify PGRN antibodies that detect single linker regions of PGRN.

FIG. 1E shows data where cell lysates were analyzed by immunoblot to identify PGRN antibodies that detect GRNs.

FIG. 2A shows sequences of recombinant granulins. Constructs contain the human PGRN signal protein followed by a twin-Strep/FLAG tag (italicized) before the GRN domains.

FIG. 2B shows sequences of recombinant granulins and linkers. Constructs contain the human PGRN signal protein followed by a twin-Strep/FLAG tag (italicized) before the GRN and linker domains.

FIG. 3A shows data indicating endogenous GRNs are found intracellularly. Whole-cell lysates (30 μg total protein) from commonly used cell types were analyzed for intracellular PGRN (R&D AF2420) and GRN (Sigma) by immunoblot.

FIG. 3B shows data where HEK-PGRN cells were grown in serum-free media for 24 hrs and the conditioned media was concentrated and analyzed for low molecular weight proteins (arrow) by coomassie stain.

FIG. 3C shows data where concentrated serum-free media from HEK-PGRN cells was analyzed for secreted cathepsin D, PGRN (R&D AF2420), and GRNs by immunoblot.

FIG. 4A shows data indicating endocytosed PGRN is rapidly processed into stable, mature GRNs. HAP1 PGRN KO cells were pulsed with mCherry-PGRN (5 μg/mL) in the media for 24 hrs and then chased with fresh media without PGRN for various lengths of time. Lysates were analyzed for PGRN (R&D AF2420) and GRN (Sigma) by immunoblot. Equal volumes of lysates were run for each time-point to account for differences in cell growth and measurements were normalized to untreated control signal.

FIG. 4B shows quantification of PGRN and GRN from experiment in FIG. 4A.

FIG. 5 shows data indicating GRNs localize to lysosomes. HeLa cells were double immunolabeled with R&D 962 AF2420 and LAMP1 antibodies. Endogenous HeLa proteins were separated by density-based gradient centrifugation and individual fractions (1-12) were analyzed for PGRN (R&D AF2420), GRN (Sigma), and organelle markers by immunoblot.

FIG. 6A shows data indicating GRN levels are regulated by SORT1 and TMEM106B expression. HAP1 PGRN KO cells were treated with C-TAP PGRN or N-TAP PGRN (5 μg/mL) for 24 hrs and 989 lysates were analyzed for PGRN and GRN by immunoblot.

FIG. 6B shows data on HAP1 WT, SORT1 KO, and PGRN KO cell lysates analyzed for endogenous levels of SORT1, PGRN, and GRN by immunoblot.

FIG. 6C shows the quantification of PGRN.

FIG. 6D shows the quantification of GRN.

FIG. 6E shows an image indicating the overexpression of TMEM106B in HeLa cells for 48 hrs results in the formation of large vacuoles. Scale bar: 20 m.

FIG. 6F shows data where HeLa cells were transfected with empty vector or TMEM106B for 48 hrs and lysates were analyzed for PGRN and GRN by immunoblot. Asterisk (*) denotes endogenous, intermediate PGRN cleavage product.

FIG. 6G shows quantification of intracellular PGRN.

FIG. 6H shows quantification of intracellular GRN.

FIG. 6I shows quantification of secreted PGRN (by ELISA).

FIG. 6J shows an image indicating overexpression of TMEM106B in HAP1 PGRN KO cells for 24 hrs results in the formation of large vacuoles. Scale bar: 10 m.

FIG. 6K shows data where HAP1 PGRN KO cells were transfected with TMEM106B for 24 hrs and then treated with mCherry-PGRN (5 μg/mL) for an additional 24 hrs. Lysates were analyzed for PGRN and GRN by immunoblot.

FIG. 6L shows quantification of PGRN.

FIG. 6M shows quantification of GRN.

FIG. 7A shows data indicating PGRN processing into GRNs is mediated by proper lysosome function and cysteine protease activity. HAP1 WT cells were treated for 24 hrs with the pan-lysosome inhibitors chloroquine (CQ; 50 μM), bafilomycin A1 (BafA1; 50 nM), or concanamycin A (ConA; 50 nM) and conditioned media was analyzed for secreted PGRN by ELISA.

FIG. 7B shows data where lysates from HAP1 WT cells treated as above were analyzed for PGRN and GRN by immunoblot. Asterisks (*) denote endogenous, intermediate PGRN cleavage products.

FIG. 7C shows data on quantification of PGRN.

FIG. 7D shows data on quantification of GRN.

FIG. 7E shows data where HAP1 WT cells were treated with the indicated protease inhibitors for 24 hrs and analyzed for PGRN and GRN by immunoblot. Asterisks (*) denote endogenous, intermediate PGRN cleavage products.

FIG. 7F shows data on time-dependent cleavage of C-TAP PGRN by recombinant cathepsin L in vitro.

FIG. 7G shows data where C-TAP PGRN was incubated with or without cathepsin L for 2 hrs in vitro and analyzed for multiple GRNs by immunoblot.

FIG. 7H shows data where HAP1 WT cells were treated with increasing concentrations of cathepsin L inhibitor II (Z-FY-CHO) for 40 hrs and lysates were analyzed for PGRN and GRN by immunoblot. Asterisks (*) denote endogenous, intermediate PGRN cleavage products.

FIG. 7I shows data on the quantification of PGRN.

FIG. 7J shows data on the quantification of GRN.

FIG. 8A shows data indicating human PGRN is processed into GRNs. PGRN KO MEF cells were treated with recombinant human PGRN (5 or 10 μg/mL) or recombinant mouse PGRN (5 μg/mL) in the media for 24 hrs and lysates were analyzed for PGRN (R&D AF2420) and GRN (Sigma) by immunoblot. R&D AF2420 displays some cross-reactivity with mouse progranulin.

FIG. 8B shows data where PGRN KO MEF cells were treated with N-TAP or C-TAP human PGRN (5 μg/mL) in the media for 24 hrs and lysates were analyzed for PGRN and GRN by immunoblot.

FIG. 8C shows data where brain tissue lysates from 14-month old GRN KO mice injected were analyzed for human PGRN (R&D AF2420) and 1039 GRNs (Sigma and Origene) by immunoblot.

FIG. 9A shows data indicating FTD patients with a GRN mutation (FTD-GRN) are haploinsufficient for GRNs. Primary fibroblast lysates from 3 control and 3 FTD-GRN patients were analyzed for PGRN and GRNs by immunoblot.

FIG. 9B shows data on the quantification of PGRN.

FIG. 9C shows the quantification of GRN-2,3 (Sigma).

FIG. 9D shows the quantification of GRN-4 (Origene).

FIG. 9E shows data where brain lysates from 5 control and 5 FTD-GRN patients were analyzed for GRNs by immunoblot.

FIG. 9F shows quantification of GRN-2,3 1050 (Sigma).

FIG. 9G shows quantification of GRN-4 (Origene).

FIG. 9H shows quantification of PGRN in the same brain lysates by ELISA.

FIG. 10 illustrates of intracellular processing of progranulin (PGRN) into stable, lysosomal granulins (GRNs). Although it is not intended that embodiments of this disclosure be limited by any particular mechanism, it is hypothesized that sortilin and other receptors target endocytosed and newly synthesized PGRN to lysosomes. Within lysosomes, PGRN is proteolytically cleaved, in part, by cysteine proteases (i.e. cathepsin L) into mature, stable GRN proteins. Ablation of sortilin results in the reduced production of GRNs. Further, lysosome dysfunction caused by alkalizing agents or TMEM106B overexpression inhibits the processing of PGRN into GRNs. FTD-GRN patients are haploinsufficient for GRNs, which may drive lysosome dysfunction leading to neurodegeneration.

FIG. 11 shows sequences comparisons of human granulin-2 (NCBI accession number 2JYV_A) SEQ ID NO: 31 to the rat (Rattus norvegicus NCBI accession number CAA44198) SEQ ID NO: 32 and the roundworm (Caenorhabditis elegans, NCBI accession number NP_492982.1) SEQ ID NO: 33. The consensus sequence SEQ ID NO: 22 is shown with conserved amino acids underlined, herein each X is any amino acid and each Z is a similar amino acid.

FIG. 12A shows data indicating GRN-2 proteins are trafficked to the lysosome and rescue the cathepsin-D (CTSD) phenotype in PGRN KO MEFs. Human recombinant PGRN and GRN-2 proteins are purified. The proteins contain a tandem affinity purification (TAP) twin Strep-tag. L3 denotes linker-3 (native C-terminal linker after GRN-2). L3+SORT denotes linker-3 plus the addition of the sortilin binding region of PGRN (QLL). Recombinant GRN-2 proteins expressed in HEK293T cells are processed into mature GRN-2 proteins.

FIG. 12B shows data where Grn KO MEF cells were treated with PGRN (10 μg/mL) or GRN-2 proteins (25 μg/mL) for 120 hrs followed by measurement of CTSD and GRN-2 levels by immunoblot.

FIG. 12C shows quantification data of total mature CTSD levels. PGRN KO cells treated with exogenous GRN2+L3+SORT protein for 48 hrs show co-localization with LAMP1-positive organelles.

FIG. 13A shows data indicating AAV-hPGRN expressed in mouse brain is processed into mature GRNs and rescues CTSD phenotype. Grn KO mice (PO) were injected with AAV vectors (eGFP or N-TAP PGRN) and analyzed for expression at 2-months of age. Data on human PGRN and GRN levels in mouse brain lysates 13 months after injection with eGFP or N-TAP PGRN is provided.

FIG. 13B shows data on quantification of CTSD levels in mouse brain lysates at 13 months.

FIGS. 14A-14D shows data demonstrating that treatment with a recombinant granulin 2 and 4 (SEQ ID NO: 14 and 16 respectively) are sufficient to rescue multiple neuropathological features in the brains of Grn−/− mice and restore the dysfunctional levels of multiple proteins back to wild-type levels. Granulins are the functional units of progranulin and delivery of a granulin to the brain of progranulin deficient (Grn−/−; Grn KO) mice ameliorates brain inflammation, lysosome dysfunction, and neuropathology. Grn KO mice are a model of neurodegenerative dementia and replicate pathologic features of frontotemporal dementia (FTD) caused by GRN mutations as well as neuronal ceroid lipofuscinosis. Treatment with a single granulin is sufficient to completely rescue multiple neuropathological features in the brains of Grn−/− mice and restore the dysfunctional levels of multiple proteins back to wild-type levels. Treatment of Grn−/− mice with either GRN2 or GRN4 ameliorates elevated CD68 and galectin-3 levels back to WT levels and is as efficacious as PGRN (positive control). Recombinant Adeno-Associated Virus (rAAV)-derived granulin-2 (GRN2) or granulin-4 (GRN4) correct disease-related neuropathology.

FIG. 14A shows data where cohorts of Grn−/− and wild-type (WT; Grn+/+) mice were injected with rAAV encoding GRN-2, GRN-4, full-length progranulin (PGRN; positive control for rescue), and GFP (negative control). Mice were aged for 12 months, then tissue harvested for analysis. Levels of autofluorescent lipofuscin were quantified in the hippocampus and thalamus of brain sections from wild-type (WT; Grn+/+) and Grn−/− mice that were injected with rAAV expressing GFP, PGRN, GRN2, and GRN4. Compared to WT mice, Grn−/− mice have higher levels of lipofuscin, a marker of lysosome damage. Treatment of Grn−/− mice with either GRN2 or GRN4 ameliorates elevated lipofuscin levels back to WT levels and is as efficacious as PGRN (positive control).

FIG. 14B shows data on levels of the glycoprotein nmb (GPNMB) that were quantified in the thalamus of brain extracts using ELISAs from wild-type (WT; Grn+/+) and Grn−/− mice that were injected with rAAV expressing GFP, PGRN, GRN2, and GRN4. Grn−/− mice have higher levels of GPNMB, which is a marker of lysosome dysfunction and lipid accumulation. Treatment of Grn−/− mice with either GRN2 or GRN4 ameliorates elevated GPNMB levels back to WT levels and is as efficacious as PGRN (positive control).

FIG. 14C shows data on levels of CD68 quantified in the thalamus of brain sections of wild-type (WT; Grn+/+) and Grn−/− mice that were injected with rAAV expressing GFP, PGRN, GRN2, and GRN4.

FIG. 14D shows data on levels of galectin-3 quantified in the thalamus of brain sections of wild-type (WT; Grn+/+) and Grn−/− mice that were injected with rAAV expressing GFP, PGRN, GRN2, and GRN4. Grn−/− mice have higher levels of CD68 and galectin-3, which are markers of microglia activation and inflammation.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

“Subject” refers to any animal, preferably a human patient, livestock, or domestic pet.

The terms “protein” and “polypeptide” refer to compounds comprising amino acids joined via peptide bonds and are used interchangeably. Amino acids may be naturally or non-naturally occurring. A “chimeric protein” or “fusion protein” is a molecule in which different portions of the protein are derived from different origins such that the entire molecule is not naturally occurring. A chimeric protein may contain amino acid sequences from the same species or different species as long as they are not arranged together in the same way that they exist in a natural state. Examples of a chimeric protein include sequences disclosed herein that are contain one, two or more amino acids attached to the C-terminal or N-terminal end that are not identical to any naturally occurring protein, such as in the case of adding an amino acid containing an amine side chain group, e.g., lysine, an amino acid containing a carboxylic acid side chain group such as aspartic acid or glutamic acid, or a polyhistidine tag, e.g. typically four or more histidine amino acids. Contemplated chimeric proteins include those with self-cleaving peptides such as P2A-GSG. See Wang. Scientific Reports 5, Article number: 16273 (2015).

A “variant” refers to a chemically similar sequence because of amino acid changes or chemical derivative thereof. In certain embodiments, a variant contains one, two, or more amino acid deletions or substitutions. In certain embodiments, the substitutions are conserved substitutions. In certain embodiments, a variant contains one, two, or ten or more an amino acid addition(s). The variant may be substituted with one or more chemical substituents.

One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both.

Variants can be tested in functional assays. Certain variants have less than 10%, and preferably less than 5%, and still more preferably less than 2% changes (whether substitutions, deletions, and so on). Variants can be tested by mutating the vector to produce appropriate codon alternatives for polypeptide translation.

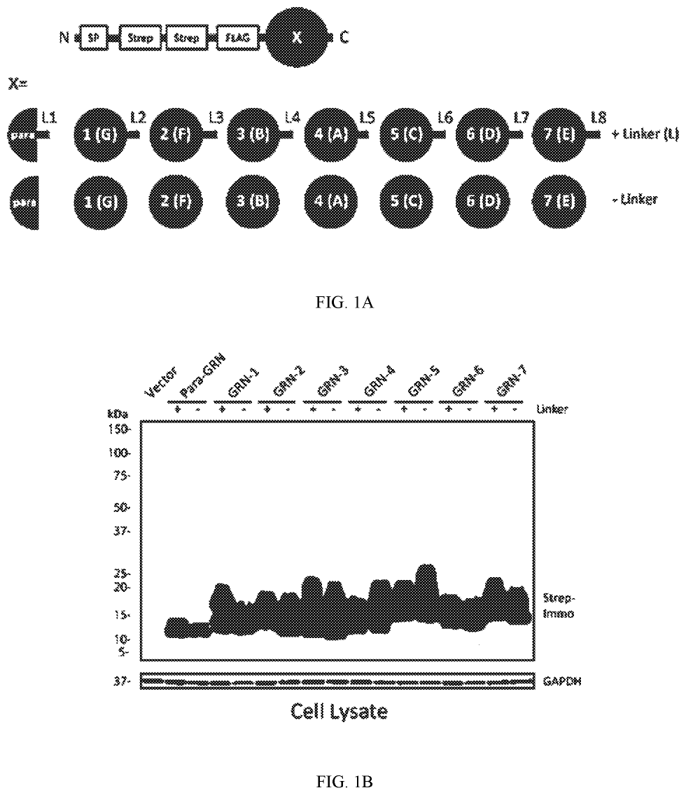

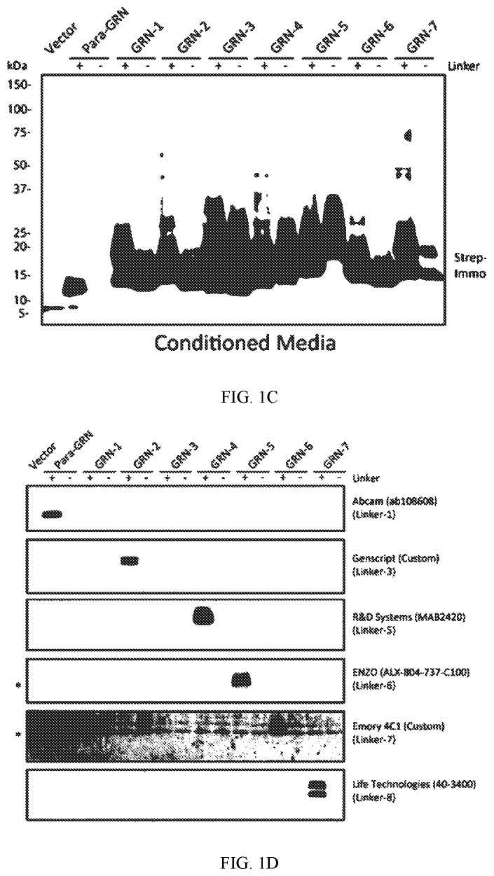

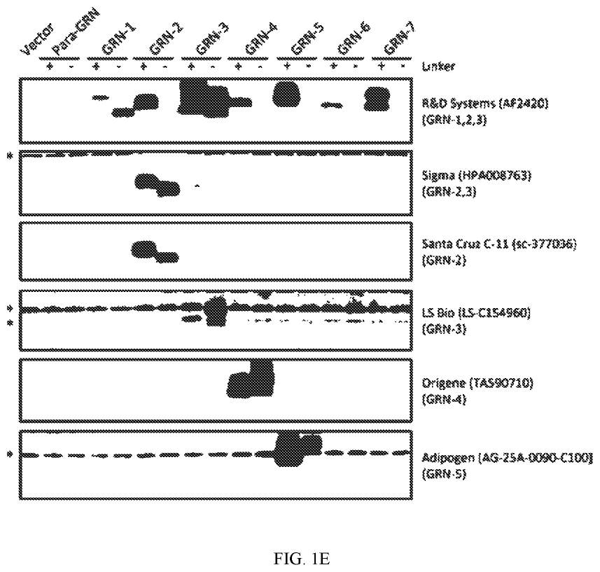

Active variants and fragments can be identified with a high probability using computer modeling. Shihab et al. report an online genome tolerance browser. BMC Bioinformatics. 2017, 18(1):20. Ng et al. report methods of predicting the effects of amino acid substitutions on protein function. Annu Rev Genomics Hum Genet. 2006, 7:61-80. Teng et al. Approaches and resources for prediction of the effects of non-synonymous single nucleotide polymorphism on protein function and interactions. Curr Pharm Biotechnol. 2008, 9(2):123-33. As pointed out on page 62 in Ng et al. (2006): “[Amino Acid Substitution (AAS)] prediction methods use sequence and/or structural information for prediction. Prediction is feasible because mutations that affect protein function tend to occur at evolutionarily conserved sites (FIG. 1a) and/or are buried in protein structure (FIG. 1b). These observations came from several early studies that used AASs found in disease genes in affected individuals (39, 68, 77). These studies assumed that these substitutions affected protein function, thereby causing disease. These studies also assumed that a majority of nsSNPs in humans or the substitutions observed between humans and closely related species are functionally neutral.”

Guidance in determining which and how many amino acid residues may be substituted, inserted, or deleted without abolishing biological activity may be found using computer programs well known in the art, for example, RaptorX, ESyPred3D, HHpred, Homology Modeling Professional for HyperChem, DNAStar, SPARKS-X, EVfold, Phyre, and Phyre2 software. See also Saldano et al. Evolutionary Conserved Positions Define Protein Conformational Diversity, PLoS Comput Biol. 2016, 12(3):e1004775; Marks et al. Protein structure from sequence variation, Nat Biotechnol. 2012, 30(11):1072-80; Mackenzie et al. Curr Opin Struct Biol. 2017, 44:161-167 Mackenzie et al. Proc Natl Acad Sci USA. 113(47):E7438-E7447 (2016); Joseph et al. J R Soc Interface. 2014, 11(95):20131147, and Wei et al. Int. J. Mol. Sci. 2016, 17(12), 2118.

As used herein, the term “derivative” refers to a structurally similar peptide that retains sufficient functional attributes of the identified analogue. The derivative may be structurally similar because it is lacking one or more atoms, e.g., replacing an amino group, hydroxyl, or thiol group with a hydrogen, substituted, a salt, in different hydration/oxidation states, or because one or more atoms within the molecule are switched, such as, but not limited to, replacing a oxygen atom with a sulfur atom or replacing an amino group with a hydroxyl group. The derivative may be a prodrug, comprise a lipid, polyethylene glycol, saccharide, polysaccharide. A derivative may be two or more peptides linked together by a linking group. It is contemplated that the linking group may be biodegradable. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

In certain embodiments, the peptides disclosed herein have at least one non-naturally occurring molecular modification, such as the attachment of polyethylene glycol, the attachment of a chimeric peptide, the attachment of a fluorescent dye comprising aromatic groups, fluorescent peptide, a chelating agent capable of binding a radionuclide such as ¹⁸F, N-terminal acetyl, propionyl group, myristoyl and palmitoyl, group or N-terminal methylation, or a C-terminal alkyl ester. In certain embodiments, the disclosure contemplates peptides disclosed herein labeled using commercially available biotinylation reagents. Biotinylated peptide can be used in streptavidin affinity binding, purification, and detection. In certain embodiments, the disclosure contemplates peptide disclose herein containing azide-derivatives of naturally occurring monosaccharides such as N-azidoacetylglucosamine, N-azidoacetylmannosamine, and N-azidoacetylgalactosamine.

In certain embodiments, this disclosure contemplates derivatives of peptide disclosed herein wherein one or more amino acids are substituted with chemical groups to improve pharmacokinetic properties such as solubility and serum half-life, optionally connected through a linker. In certain embodiments, such a derivative may be a prodrug wherein the substituent or linker is biodegradable, or the substituent or linker is not biodegradable. In certain embodiments, contemplated substituents include a saccharide, polysaccharide, acetyl, fatty acid, lipid, and/or polyethylene glycol. The substituent may be covalently bonded through the formation of amide bonds on the C-terminus or N-terminus of the peptide optionally connected through a linker. In certain embodiments, it is contemplated that the substituent may be covalently bonded through an amino acid within the peptide, e.g. through an amine side chain group such as lysine or an amino acid containing a carboxylic acid side chain group such as aspartic acid or glutamic acid, within the peptide comprising a sequence disclosed herein. In certain embodiments, it is contemplated that the substituent may be covalently bonded through a cysteine in a sequence disclosed herein optionally connected through a linker. In certain embodiments, a substituent is connected through a linker that forms a disulfide with a cysteine amino acid side group.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl. The substituents may further optionally be substituted.

As used herein, a “lipid” group refers to a hydrophobic group that is naturally or non-naturally occurring that is highly insoluble in water. As used herein a lipid group is considered highly insoluble in water when the point of connection on the lipid is replaced with a hydrogen and the resulting compound has a solubility of less than 0.63×10⁻⁴% w/w (at 25° C.) in water, which is the percent solubility of octane in water by weight. See Solvent Recovery Handbook, 2^{nd} Ed, Smallwood, 2002 by Blackwell Science, page 195. Examples of naturally occurring lipids include saturated or unsaturated hydrocarbon chains found in fatty acids, glycerolipids, cholesterol, steroids, polyketides, and derivatives. Non-naturally occurring lipids include derivatives of naturally occurring lipids, acrylic polymers, aromatic, and alkylated compounds and derivatives thereof.

The term “prodrug” refers to an agent that is converted into a biologically active form in vivo. Prodrugs are often useful because, in some situations, they may be easier to administer than the parent compound. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto (thiol) group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

For example, if a disclosed peptide or a pharmaceutically acceptable form of the peptide contains a carboxylic acid functional group, a prodrug can comprise a pharmaceutically acceptable ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as beta-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

If a disclosed peptide or a pharmaceutically acceptable form of the peptide contains an alcohol functional group, a prodrug can be formed by the replacement of the hydrogen atom of the alcohol group with a group such as (C₁-C₆)alkanoyloxymethyl, 1-((C₁-C₆)alkanoyloxy) ethyl, 1-methyl-1((C₁-C₆)alkanoyloxy)ethyl (C₁-C₆)alkoxycarbonyloxymethyl, —N—(C₁-C₆)alkoxycarbonylaminomethyl, succinoyl, (C₁-C₆)alkanoyl, alpha-amino(C₁-C₄)alkanoyl, arylacyl and alpha-aminoacyl, or alpha-aminoacyl-alpha-aminoacyl, where each alpha-aminoacyl group is independently selected from naturally occurring L-amino acids P(O)(OH)₂, —P(O)(O(C₁-C₆)alkyl)₂, and glycosyl (the radical resulting from the removal of a hydroxyl group of the hemiacetal form of a carbohydrate).

If a disclosed peptide or a pharmaceutically acceptable form of the peptide incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural alpha-aminoacyl, —C(OH)C(O)OY₁ wherein Y¹ is H, (C₁-C₆)alkyl or benzyl, —C(OY₂)Y₃ wherein Y₂ is (C₁-C₄) alkyl and Y₃ is (C₁-C₆)alkyl, carboxy(C₁-C₆)alkyl, amino(C₁-C₄)alkyl or mono-Nor di-N,N—(C₁-C₆)alkylaminoalkyl, —C(Y₄)Y₅ wherein Y₄ is H or methyl and Y₅ is mono-N- or di-N,N—(C₁-C₆)alkylamino, morpholino, piperidin-1-yl or pyrrolidin-1-yl.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

As used herein, “pharmaceutically acceptable enol ethers” include, but are not limited to, derivatives of formula —C═C(OR) where R can be selected from alkyl, alkenyl, alkynyl, aryl, aralkyl, and cycloalkyl. Pharmaceutically acceptable enol esters include, but are not limited to, derivatives of formula —C═C(OC(O)R) where R can be selected from hydrogen, alkyl, alkenyl, alkynyl, aryl, aralkyl, and cycloalkyl.

A “linking group” refers to any variety of molecular arrangements that can be used to bridge to molecular moieties together. An example formula may be —Rₘ— wherein R is selected individually and independently at each occurrence as: —CRₘRₘ—, —CHRₘ—, —CH—, —C—, —CH₂—, —C(OH)Rₘ, —C(OH)(OH)—, —C(OH)H, —C(Hal)Rₘ—, —C(Hal)(Hal)-, —C(Hal)H—, —C(N₃)Rₘ—, —C(CN)Rₘ—, —C(CN)(CN)—, —C(CN)H—, —C(N₃)(N₃)—, —C(N₃)H—, —O—, —S—, —N—, —NH—, —NRₘ—, —(C═O)—, —(C═NH)—, —(C═S)—, —(C═CH₂)—, which may contain single, double, or triple bonds individually and independently between the R groups. If an R is branched with an Rₘ it may be terminated with a group such as —CH₃, —H, —CH═CH₂, —CCH, —OH, —SH, —NH₂, —N₃, —CN, or -Hal, or two branched Rs may form a cyclic structure. It is contemplated that in certain instances, the total Rs or “m” may be less than 100, or 50, or 25, or 10. Examples of linking groups include bridging alkyl groups and alkoxyalkyl groups. Linking groups may be substituted with one or more substituents.

As used herein, the term “biodegradable” in reference to a substituent or linker refers to a molecular arrangement in a peptide derivative that when administered to a subject, e.g., human, will be broken down by biological mechanism such that a metabolite will be formed and the molecular arrangement will not persist for over a long period of time, e.g., the molecular arrangement will be broken down by the body after a several hours or days. In certain embodiments, the disclosure contemplates that the biodegradable linker or substituent will not exist after a week or a month.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “sterilized” refers to subjecting something to a process that effectively kills or eliminates transmissible agents (such as fungi, bacteria, viruses, prions and spore forms etc.). Sterilization can be achieved through application of heat, chemicals, irradiation, high pressure or filtration. One process involves water prepared by distillation and stored in an airtight container wherein suitable additives are introduced to approximate isotonicity.

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements.

A “heterologous” nucleic acid sequence or peptide sequence refers to a nucleic acid sequence or peptide sequence that do not naturally occur, e.g., because the whole sequences contain a segment from other plants, bacteria, viruses, other organisms, or joinder of two sequences that occur the same organism but are joined together in a manner that does not naturally occur in the same organism or any natural state.

As used herein, “operably linked” refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occur in nature. The segments may be joined in an altered arrangement such that the entire nucleic acid sequence from start to finish does not naturally occur. The term “recombinant” when made in reference to a protein or a polypeptide refers to a protein molecule that is expressed using a recombinant nucleic acid molecule.

As used herein, “purified” means separated from other compounds or entities. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo and in vitro (cell free) systems. Systems for recombinant protein expression typically utilize cells transfecting with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme that confers a blue color in the presence of X-gal (5-bromo-4-chloro-3-indolyl-o-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: amp^{r}, cam^{r}, tet^{r}, blasticidin^{r}, neo^{r}, hyg^{r}, abx^{r}, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose:galactose-1-phosphate uridyltransferaseI (galT), feedback-insensitive a subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB1), dehalogenase (dhlA), mannose-6-phosphate reductase gene (M6PR), hygromycin phosphotransferase (HPT), and D-serine ammonialyase (dsdA).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous polypeptide in the receptor. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, the disclosure relates to granulins comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a polypeptide disclosed herein or chimeric protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

In certain embodiments, term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG (SEQ ID NO: 36) and GGGGT (SEQ ID NO: 37) have a sequence identity of 4 out of 5 or 80%. For example, the polypeptides GGGPPP (SEQ ID NO: 38) and GGGAPPP (SEQ ID NO: 39) have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L M F G P; Charged positive: R K H; Charged negative—D E; Polar—C S T N Q N Y W H; hydrogen bonding carboxylic acid or carboxamide—D, E, N, and Q. The amino acid groups are also considered conserved substitutions: Aromatic—F Y W; hydrophobic—A V I L; Charged positive: R K H; Charged negative—D E; Polar—S T N Q.

### Granulins are Stable Lysosomal Proteins

GRN mutations cause FTD through haploinsufficiency or loss of function of PGRN. However, it is unknown how GRN mutations affect levels of GRNs in the brain, and it is unclear why loss of PGRN in the brain causes neurodegeneration. Although it is not intended that embodiments of this disclosure be limited by any particular mechanism, it is hypothesized that PGRN haploinsufficiency causes lysosome dysfunction. Lysosome dysfunction is a common occurrence in numerous neurodegenerative diseases. Prior reports indicate that GRNs are produced from extracellular PGRN. Experiments reported herein indicate that GRNs are produced intracellularly in the lysosome.

The identification and characterization of antibodies that reliably detect human GRNs by immunoblot and immunocytochemistry are reported herein. Mature GRNs are stably produced from PGRN and localize to LAMP1-positive lysosomes. The transmembrane protein 106B (TMEM106B), a neuronal lysosomal protein that regulates lysosome transport and function, is a strong genetic risk factor for FTD-GRN. The generation of GRNs from PGRN is inhibited by SORT1 depletion, pan-lysosomal inhibitors, or expression of the FTD-GRN modifier TMEM106B. The proteolytic processing of PGRN into GRNs, mediated in part by cysteine protease activity, is conserved between humans and mice. Endogenous levels of multiple GRNs are haploinsufficient in FTD-GRN patient fibroblasts and brain mirroring full-length PGRN. These studies indicate that lysosomal deficiency of GRNs may initiate lysosome dysfunction contributing to neurodegeneration.

Each of the human GRN proteins were generated with and without their carboxyl-terminal linker regions (FIGS. 1A, 1B, and 1C). Using these constructs, the epitopes of anti-PGRN antibodies were determined using immunoblots. Multiple antibodies were identified that detect individual linker regions of PGRN as well as antibodies that recognize GRN-1, GRN-2, GRN-3, GRN-4, and GRN-5 by immunoblot (FIGS. 1C and 1D).

A human GRN knock-out cell line was developed using CRISPR-Cas9 technology (HAP1 PGRN KO). A stable cell line was developed that overexpresses human PGRN (HEK-PGRN) to aid in the screening and specificity of PGRN antibodies for detection of GRNs. These two cell lines are used to verify whether antibodies recognize specific PGRN cleavage products.

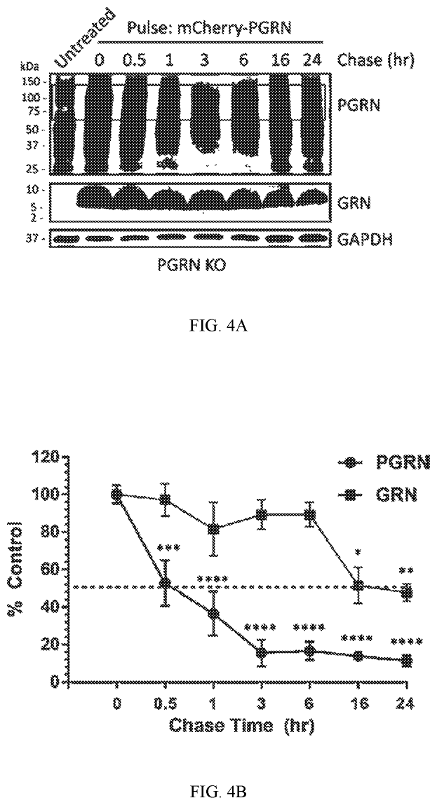

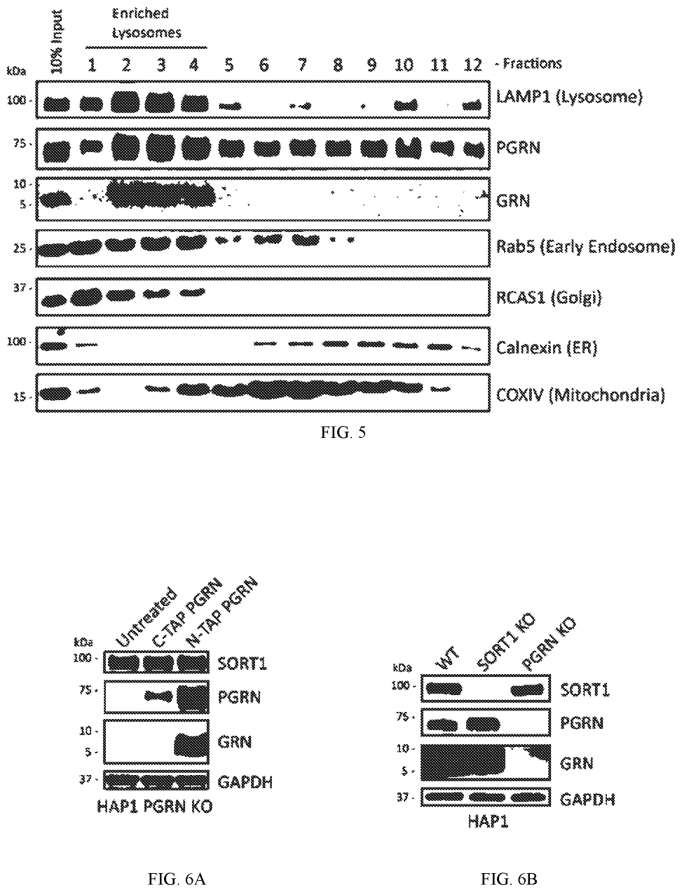

FTD-GRN patients have half the normal levels of PGRN and abnormal accumulation of lysosomal proteins, enzymes, and storage material which is recapitulated in Grn KO mice. Further, a mutation in both copies of GRN in humans causes a lysosomal storage disease called neuronal ceroid lipofuscinosis (NCL; CLN11). Finally, PGRN is shuttled to the lysosome via SORT1 or PSAP dependent pathways in cells and tissue. PGRN pulse-chase assay was developed to study the generation, localization, and stability of GRNs (FIG. 4B). Using antibodies that detect GRNs and our PGRN KO cell line, endocytosed PGRN is rapidly processed into mature, stable GRNs that localize to LAMP1-positive lysosomes (FIG. 5). This is in line with the original description of GRNs (A.K.A. epithelins) as heat- and acid-stable proteins. Thus, the GRNs, rather than PGRN, likely regulate critical lysosome functions.

The striking parallel between PGRN and PSAP strengthens the hypothesis for a functional role of GRNs in lysosomes. First, PGRN and PSAP interact with each other and with SORT1. PSAP, like PGRN, is processed into smaller lysosomal proteins called saposins (SAPs A, B, C, and D). The individual SAPs have roles in lysosomal hydrolase activation needed for lipid metabolism and degradation. Deficiency in PSAP or individual SAPs leads to several lysosomal storage disorders including Gaucher disease, Krabbe disease, and metachromatic leukodystrophy. Similarly, deficient levels of GRNs may cause lysosomal dysfunction underlying FTD or NCL neurodegeneration.

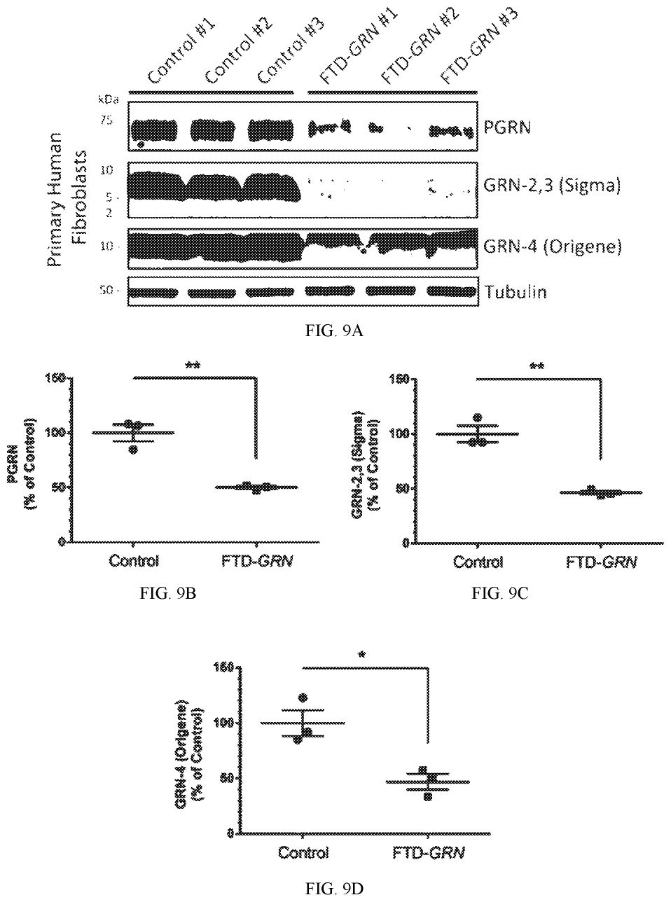

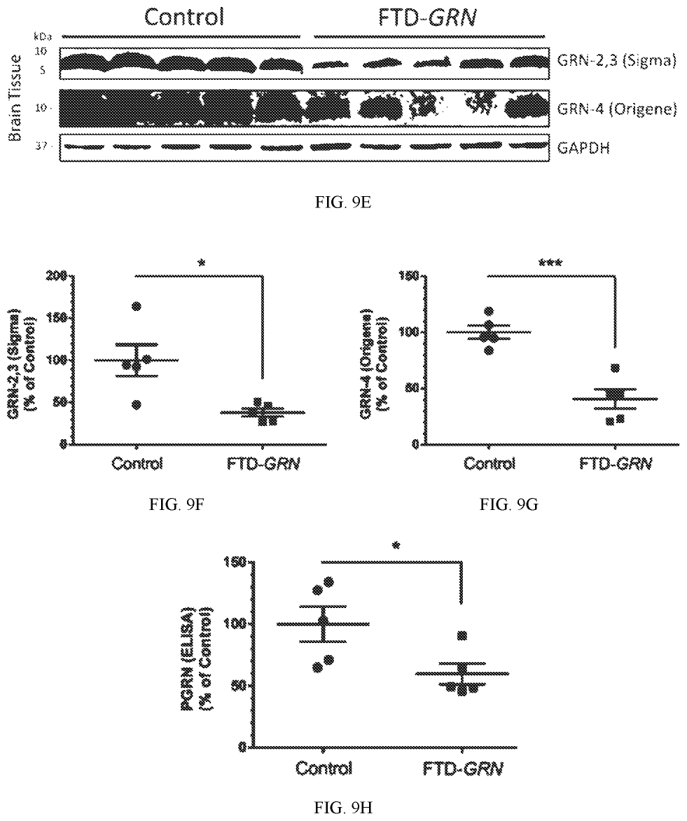

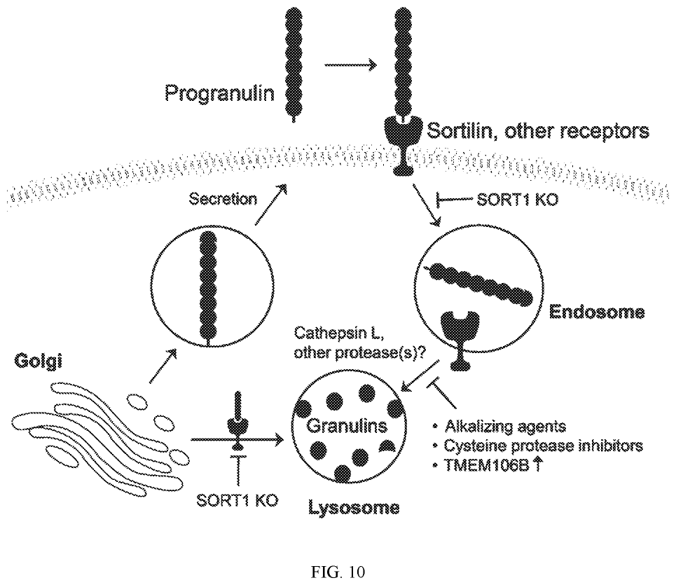

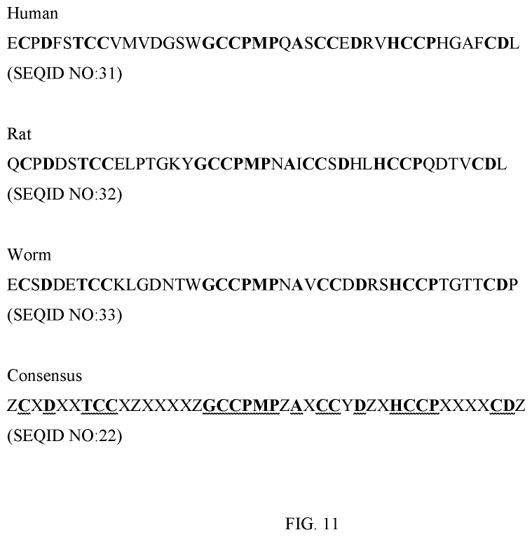

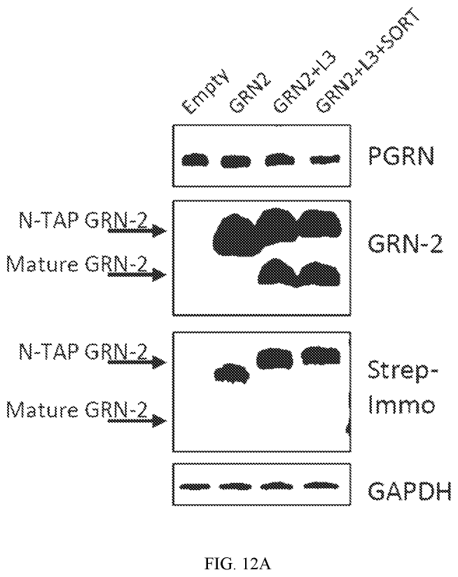

Increased production of GRNs may contribute to neurodegenerative disease. An approximately 33 kDa PGRN intermediate fragment accumulates in diseased regions of Alzheimer's disease (AD) or FTD-TDP patient brains using an antibody generated against GRN-7(E) (Salazar et al., 2015). A defect in processing of PGRN to fully mature GRNs may be caused by lysosome dysfunction. Studies reported herein indicate multiple GRNs are decreased by approximately half in both FTD-GRN primary cells and brain tissue (FIG. 9).

Experiments herein indicate that PGRN is trafficked to the lysosome based on our observation that a) deletion of SORT1 does not eliminate production of GRNS and b) recombinant PGRN that is unable to bind SORT1 (C-TAP PGRN) is internalized by MEFs and processed into GRNs. Because lysosome dysfunction is a hallmark of many neurodegenerative diseases, deficient production of GRNs may be a common contributor to the disease process. Thus, treating subject with GRNs, particularly GRN-2, is thought to be helpful for managing neurodegenerative diseases. Indeed, reduced levels of PGRN have been linked to Alzheimer's disease (Kelley et al., Archives of Neurology 67:171-177, 2010; Lee et al., Neuro-degenerative Diseases 8:216-220, 2011; Minami et al., Nature medicine 20:1157-1164, 2014; Sheng et al., Gene 542:141-145, 2014) and Parkinson's disease (Chang et al., PLoS One 8:e54448, 2013; Mateo et al., J European Federation of Neurological Societies 20:1571-1573 2013; Van Kampen et al., PLoS One 9:e97032, 2014; Chen et al., J Neurology 262:814-822 2015).

### Pharmaceutical Methods and Compositions

Methods of administering granulins disclosed herein, derivatives, variants, or vectors encoding the same include, but are not limited to, parenteral administration (e.g., intradermal, intramuscular, intraperitoneal, intravenous and subcutaneous), epidural, and mucosal (e.g., intranasal and oral routes). In a specific embodiment, the granulins or chimeric proteins are administered by temporal vein injection (intravenous) injection, injecting directly into cerebral lateral ventricles (intracerebroventricular), or by direct injection, implantation or exposure into the brain through pressure driven infusion (convection-enhanced delivery). The compositions may be administered together with other biologically active agents.

In certain embodiments, pulmonary administration can also be employed, e.g., by use of an inhaler or nebulizer, and formulation with an aerosolizing agent. See, e.g., U.S. Pat. Nos. 6,019,968; 5,985, 20; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In certain embodiments, the aerosolizing agent or propellant is a hydrofluoroalkane, 1,1,1,2-tetrafluoroethane, 1,1,1,2,3,3,3-heptafluoropropane, propane, n-butane, isobutene, carbon dioxide, air, nitrogen, nitrous oxide, dimethyl ether, trans-1,3,3,3-tetrafluoroprop-1-ene, or combinations thereof.

In a specific embodiment, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers.

The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. For granulins and fusion proteins, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Further, the dosage and frequency of administration of proteins may be reduced by enhancing uptake and tissue penetration of the fusion proteins by modifications such as, for example, lipidation.

In certain embodiments, compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. In certain embodiments, the pharmaceutical compositions contain a pharmaceutically acceptable excipient that is a solubilizing agent such as a lipid, cholesterol, fatty acid, fatty acid alkyl ester, linoleic acid, oleic acid arachidonic acid, saccharide, polysaccharide, cyclodextrin, 2-hydoxypropyl(cyclodextrin), or combinations thereof.

In certain embodiments, the pharmaceutical compositions are in solid form surrounded by an enteric coating, i.e., a polymer barrier applied on oral medication that prevents its dissolution or disintegration in the gastric environment. Compounds typically found in enteric coatings include methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, and combinations thereof.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

One embodiment provides a pharmaceutical pack or kit comprising one or more containers filled with granulins disclosed herein or vectors encoding the same. Additionally, one or more other prophylactic or therapeutic agents useful for the treatment of a disease can also be included in the pharmaceutical pack or kit. One embodiment provides a pharmaceutical pack or kit including one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

In certain embodiment, this disclosure contemplates pharmaceutical compositions comprising proteins disclosed herein and pharmaceutically acceptable excipient. In certain embodiments, this disclosure contemplates the production of a medicament comprising proteins disclosed herein and uses for methods disclosed herein.

In certain embodiments, the disclosure relates to pharmaceutical compositions comprising granulins disclosed herein or vectors encoding the same and a pharmaceutically acceptable excipient. In certain embodiments, the composition is a pill or in a capsule or the composition is an aqueous buffer, e.g., a pH between 6 and 8. In certain embodiments, the pharmaceutically acceptable excipient is selected from a filler, glidant, binder, disintegrant, lubricant, and saccharide.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil and viscoleo) and injectable organic esters such as ethyl oleate.

Prevention of the action of microorganisms may be controlled by addition of any of various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the proteins may be admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Liquid dosage forms for oral administration include pharmaceutically acceptable emulsions, solutions, suspensions, syrups, and elixirs. In addition to the proteins, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

In certain embodiments, production processes are contemplated which two components, granulins disclosed herein or vectors encoding the same and a pharmaceutical carrier, are provided already in a combined dry form ready to be reconstituted together. In other embodiments, it is contemplated that proteins disclosed herein and a pharmaceutical carrier are admixed to provide a pharmaceutical composition.

Providing a pharmaceutic composition is possible in a one-step process, simply by adding a suitable pharmaceutically acceptable diluent to the composition in a container. In certain embodiments, the container is preferably a syringe for administering the reconstituted pharmaceutical composition after contact with the diluent. In certain embodiments, the coated proteins can be filled into a syringe, and the syringe can then be closed with the stopper. A diluent is used in an amount to achieve the desired end-concentration. The pharmaceutical composition may contain other useful component, such as ions, buffers, excipients, stabilizers, etc.

A “dry” pharmaceutical composition typically has only a residual content of moisture, which may approximately correspond to the moisture content of comparable commercial products, for example, has about 12% moisture as a dry product. Usually, the dry pharmaceutical composition according to the present invention has a residual moisture content preferably below 10% moisture, more preferred below 5% moisture, especially below 1% moisture. The pharmaceutical composition can also have lower moisture content, e.g. 0.1% or even below. In certain embodiments, the pharmaceutical composition is provided in dry in order to prevent degradation and enable storage stability.

A container can be any container suitable for housing (and storing) pharmaceutically compositions such as syringes, vials, tubes, etc. The pharmaceutical composition may then preferably be applied via specific needles of the syringe or via suitable catheters. A typical diluent comprises water for injection, and NaCl (preferably 50 to 150 mM, especially 110 mM), CaCl₂) (preferably 10 to 80 mM, especially 40 mM), sodium acetate (preferably 0 to 50 mM, especially 20 mM) and mannitol (preferably up to 10% w/w, especially 2% w/w). Preferably, the diluent can also include a buffer or buffer system so as to buffer the pH of the reconstituted dry composition, preferably at a pH of 6.2 to 7.5, especially at pH of 6.9 to 7.1.

In certain embodiments, the diluent is provided in a separate container. This can preferably be a syringe. The diluent in the syringe can then easily be applied to the container for reconstitution of the dry compositions. If the container is also a syringe, both syringes can be finished together in a pack. It is therefore preferred to provide the dry compositions in a syringe, which is finished with a diluent syringe with a pharmaceutically acceptable diluent for reconstituting, said dry and stable composition.

In certain embodiments, this disclosure contemplates a kit comprising a pharmaceutical composition disclosed herein and a container with a suitable diluent. Further components of the kit may be instructions for use, administration means, such as syringes, catheters, brushes, etc. (if the compositions are not already provided in the administration means) or other components necessary for use in medical (surgical) practice, such as substitute needles or catheters, extra vials or further wound cover means. In certain embodiments, the kit comprises a syringe housing the dry and stable hemostatic composition and a syringe containing the diluent (or provided to take up the diluent from another diluent container).

### Examples

### Cloning of Human GRN Expression Vectors

The DNA sequences for individual GRNs were synthesized. First, the amino acid sequence for human para-GRN and each GRN (1 through 7) including the linker region at the carboxyl-terminal end was identified based on the Universal Protein Resource database (P287991; GRN_HUMAN). The endogenous PGRN signal peptide (SP) sequence followed by a twin-Strep and FLAG tag was added to the amino-terminus of each GRN. Synthetic GRN gene constructs were designed to add a 5′ HindIII (AAGCTT) (SEQ ID NO: 40) site, a Kozak sequence (GCCACC) (SEQ ID NO: 41) before the ATG start codon, a 3′ Stop codon, and a XhoI (TGACTCGAG) (SEQ ID NO: 42) site. Following synthesis, each gene was inserted into the pcDNA3.1 (+) vector using a HindIII/XhoI cloning strategy. All constructs were verified using DNA sequencing, restriction digests, and PCR amplification. Subsequently, primers were designed for each GRN to remove the linker region; each construct was amplified via PCR, and subcloned into pcDNA3.1 (+) vector, and verified using DNA sequencing.

### Purification of Recombinant PGRNs

A tandem affinity purification (TAP) tag was cloned onto the carboxyl (C) terminus or amino (N) terminus of full-length human PGRN to generate C-TAP PGRN or N-TAP PGRN. C-TAP PGRN contains a twin-Strep tag followed by the FLAG epitope. N-TAP PGRN contains a twin Strep tag followed by a V5™ epitope tag inserted following the endogenous PGRN signal protein sequence. Stable HEK 293T cell lines overexpressing either C-TAP PGRN or N-TAP PGRN were generated. Stable cells were cultured and maintained in DMEM that contained 100 μg/ml Zeocin™ (Life Technologies™/Thermo-Fisher™; Carlsbad, CA) and conditioned media was collected. PGRN was affinity-purified from conditioned media over Strep-Tactin™ XT Superflow (Cat.no: 2-4010-025) resin using a slightly modified protocol as described by the manufacturer (IBA™ GmbH; Gottingen, Germany). The mCherry™-PGRN construct has been described (Hu et al., 2010). HEK Expi293™ cells (RRID:CVCL_D615) were transfected with the mCherry™-PGRN construct and conditioned media was collected following the manufacturer's protocol (Thermo-Fisher™; Cat #A14635). mCherry™-PGRN contains a poly-histidine tag and was purified from the media over a His-Tag column following manufacturer's protocol (Sigma-Aldrich™; Cat.no: 05893682001). For all purifications, elutions containing recombinant PGRNs were concentrated and desalted into PBS using Vivaspin™ 500 Protein Concentrators (molecular weight cut-off 50 kDa; Cat.no: 28932218; GE Healthcare Life Sciences™). The purity of recombinant PGRN was assessed by SDS-PAGE followed by colloidal coomassie dye G-250 protein stain (GelCode™ Blue; Thermo-Fisher™) and estimated to be greater than 95% pure.

### Transfections

Typical production schedule: Split cells from growth media. Transfect cells with pF Delta 6™, pH2™, and pAVV plasmids, and polyethylenimine (PEI). Harvest cells and process, lyse by freeze thaw. Store at −20 C. Isolate virus on iodixanol gradient and uncoat virus for PCR. Run PCR using primers bind to a region of the WPRE. Denature capsid proteins and perform gel analysis of proteins.

The TMEM106B constructs (AAV1 backbone; untagged or with C-terminal V5′ tag) have been described by Nicholson. See J Neurochem 126:781-7912013. HeLa or HAP1 PGRN KO cells were plated in 6-well plates and transfected with 2 μg of empty vector or TMEM106B for a total of 48 hrs. For the PGRN KO cells, 24 hrs after transfection, mCherry™-PGRN (5 μg/mL) was added to the media for an additional 24 hrs before lysing the cells for SDS-PAGE and immunoblot analysis.

### Human AAV2/1-PGRN Injections

Somatic brain transgenesis (SBT) were performed to overexpress eGFP or human N-TAP PGRN in Grn KO mice of either sex. Briefly, recombinant adeno-associated virus serotype 2/1 (AAV2/1) encoding eGFP or human N-TAP PGRN were generated and neonatal PO injections of rAAV2/1 were performed. See Chakrabarty et al., PLoS ONE 8:e67680, 2013.

Expression of Granulin Proteins and Identification of Antibodies that Detect Granulins

The lack of tools to study endogenous GRNs has limited the field's ability to generate a complete understanding how PGRN loss leads to lysosome dysfunction and neurodegeneration. To overcome this gap, a system was developed to selective express each GRN in mammalian cells, which enabled one to screen anti-human PGRN antibodies by immunoblot to determine which region(s) of PGRN they detect. Expression constructs were generated for each of the human GRN proteins: Para-GRN, GRN-1, GRN-2, GRN-3, GRN-4, GRN-5, GRN-6, and GRN-7, with (+) and without (−) their endogenous carboxyl-terminus linker regions (FIG. 1A). At the amino-terminus, each GRN construct was engineered to contain the authentic PGRN signal protein to ensure proper routing into the secretory pathway, followed by a twin-Strep and FLAG™ tag, to facilitate purification and detection. Next, the GRN constructs were transfected into HEK293T cells to detect all of the GRN proteins in cell lysates and conditioned media, indicating GRNs are properly synthesized and secreted (FIGS. 1B and 1C). Some GRNs appeared as multiple bands, suggesting post-translational glycosylation. Next, this expression system was used to screen a panel of commercial and in-house polyclonal and monoclonal antibodies developed against recombinant full-length PGRN or peptides using immunoblot. Many of the PGRN antibodies screened detect a single linker region of PGRN suggesting that the unstructured linker regions in PGRN may be immuno-dominant epitopes (FIG. 1D). Encouragingly, multiple antibodies were identified that detect one or more specific GRNs (FIG. 1E). These included the AF2420™ (R&D Systems™) polyclonal antibody, which detects GRNs-1, -2 (weakly), and -3 and antibodies that detect GRN-2 (Sigma™, Santa Cruz C-11), GRN-3 (LS Bio™, Sigma weakly), GRN-4 (Origene™), and GRN-5 (Adipogen™). Notably, the AF2420™ antibody detects multiple linker regions of PGRN, in addition to GRNs, making it a potentially useful tool for dissecting intermediate PGRN cleavage products in combination with other linker-specific antibodies.

### Endogenous, Intracellular GRNs are Present in Various Cell Types

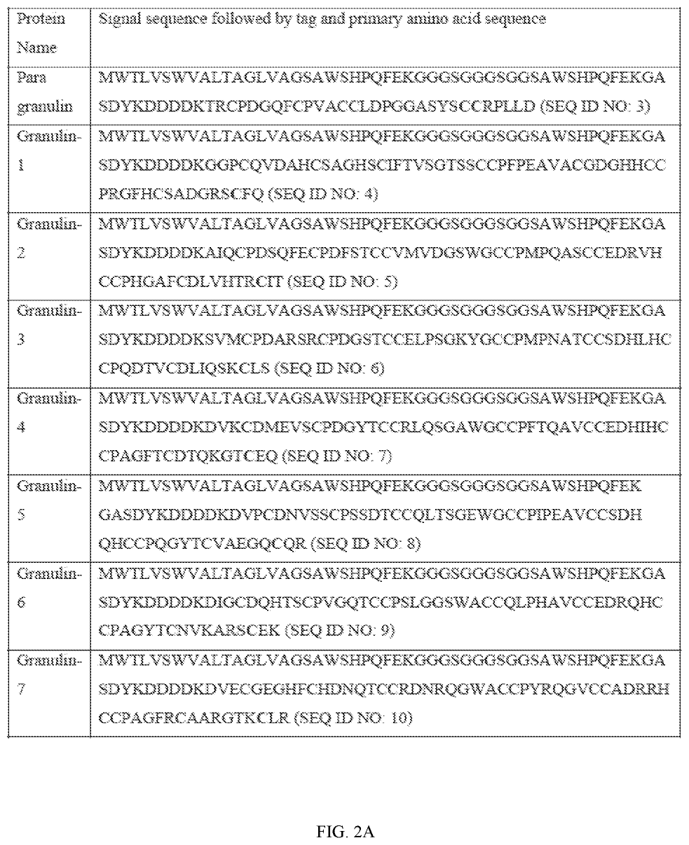

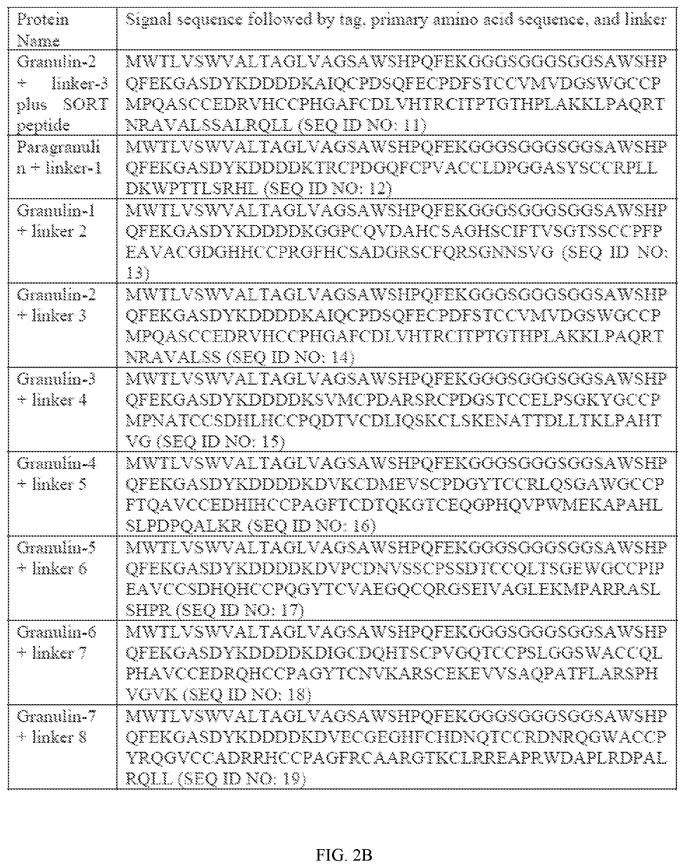

Next, experiments were performed to determine if any of the PGRN antibodies could detect endogenous GRNs in cell lysates by immunoblot. To test antibody specificity, GRN was knocked out in a near-haploid human cell line using CRISPR-Cas9 to generate the HAP1 PGRN KO cell line. Then, antibody reactivity was compared in whole-cell lysates from HAP1 wild-type (WT) and PGRN KO lines. Most antibodies detected endogenous, full-length PGRN in HAP1 WT lysates at the expected molecular weight of −75 kDa (FIG. 2A). In addition, multiple immunoreactive bands in the 15 to 75 kDa range were observed in both WT and PGRN KO cells indicating they are non-specific. Two antibodies (Sigma™ and Origene™) detected specific bands at lower molecular weights (<15 kDa) that correspond to the expected size of individual mature GRN proteins. Next, the same panel of PGRN antibodies were screened against immunoblots of whole-cell lysates from HEK293T cells stably overexpressing human PGRN (HEK-PGRN). Multiple antibodies (R&D AF2420™, Sigma™, Santa Cruz™ C-11, Origene™) were identified that detected specific bands at the expected molecular weights for mature GRNs that were increased in the HEK-PGRN lysates compared to HEK-WT lysates. GRNs were only detected in the reduced samples (+TCEP), whereas full-length PGRN could often be detected in reduced or non-reduced samples. Endogenous GRN bands <15 kDa in the HEK-PGRN lysates were not detect using the antibodies specific for GRN-3 (LS Bio™) or GRN-5 (Adipogen™). Together, these data indicate that multiple GRN proteins, specifically GRN-2 and GRN-4, are endogenously present inside cells. Finally, the specificity of the PGRN antibodies were assessed using fluorescent immunocytochemistry in HAP1 WT and PGRN KO cells. Only the R&D MAB2420™ and R&D AF2420™ antibodies were specific for PGRN/GRN labeling in these cells. The non-specific staining of the other PGRN antibodies mirrors the non-specificity that was observed by immunoblot.

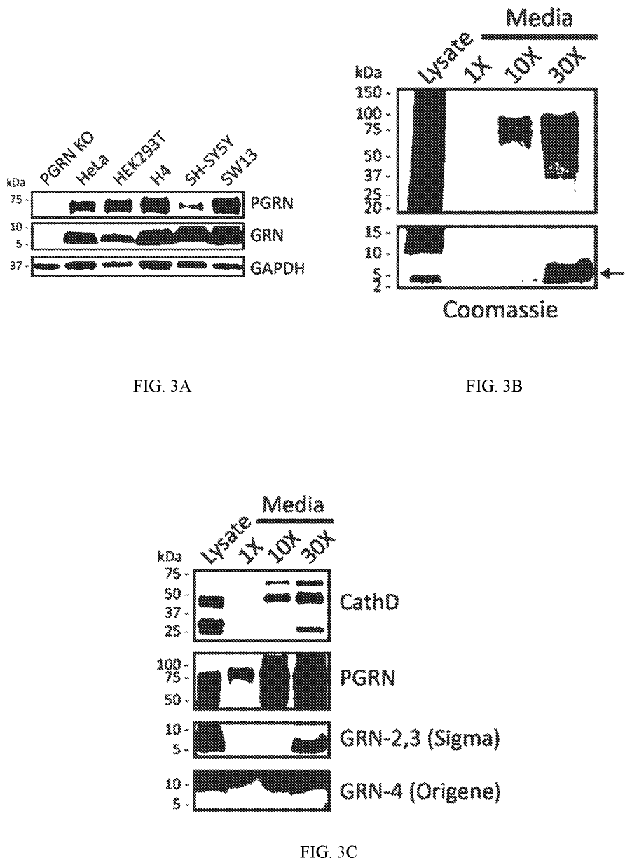

Based on our observations, the Sigma antibody was the most sensitive and reliable for detection of human GRN by immunoblot. This antibody strongly recognizes GRN-2, with weaker reactivity to GRN-3 (see FIG. 1D). Therefore, this antibody was used to further study endogenous GRN expression and production. GRN could be detected at steady-state levels in all human cell lines tested, notably with the highest levels in H4 (neuroglioma) and SH-SY5Y (neuroblastoma) cell lines (FIG. 3A). To determine if GRNs can be detected extracellularly, HEK-PGRN cells were grown in serum-free media (Opti-MEM) for 24 hours, the media was concentrated in a centrifugal spin filter (3 kDa molecular weight cut-off), and the concentrate were analyzed by coomassie total protein stain and immunoblot. A protein band (about 5 kDa) was detected in the media using coomassie staining that increased in intensity with concentration (FIG. 3B). This band is likely insulin (MW: 5.8 kDa), which is a component of Opti-MEM media and demonstrates that low molecular weight proteins were enriched in the concentrate. By immunoblot, robust levels of secreted PGRN and cathepsin D were verified, which served as a positive control for a secreted lysosomal protein, in the concentrated media. Conversely, GRNs were barely detectable, even in the 30× concentrated media sample (FIG. 3C). These results indicate that GRNs are not constitutively secreted or generated from extracellular PGRN in cultured cells.

### GRNs are Stable Lysosomal Proteins

To monitor the production, stability, and localization of GRNs inside cells, a PGRN pulse-chase assay was developed based on the observation that application of purified extracellular PGRN was rapidly endocytosed by many cell lines. HAP1 GRN KO cells were treated (pulsed) with human mCherry™-PGRN for 24 hours and then chased with fresh media without PGRN for varying lengths of time. The levels of PGRN (R&D AF2420™) and GRN (Sigma™) were measured at each timepoint by immunoblot. After a 24 hour pulse of mCherry™-PGRN, robust intracellular PGRN and GRN signals were observed by immunoblot (FIG. 4A). After only 30 minutes of chase, approximately 50% of the PGRN signal was lost and by 6 hours it was virtually absent (FIG. 4B). Strikingly, the GRN signal remained stable with an estimated half-life of at least about 16 hours, with detection out to 24 hours (FIGS. 4A and 4B). These data indicate that endocytosed PGRN is rapidly processed into mature, stable GRNs. Similar results were obtained using N-TAP PGRN in the pulse-chase assay. Accumulation of intermediate PGRN fragments were not observe in the ˜15-60 kDa range at any time point during the pulse-chase assay using the R&D AF2420™ antibody which detects multiple linker regions of PGRN (FIG. 4A).

Next, PGRN KO cells pulse-chased with mCherry™-PGRN (24 hr pulse/6 hr chase) were immunostained. Following the 6 hour chase, the R&D AF2420™ antibody still gave a robust immunofluorescent signal. At this time point (t=6 hr) GRN, but not PGRN, was detect by immunoblot (see FIG. 4A). As a control, another set of cells were stained with the R&D MAB2420™ antibody which is linker specific and only recognizes full-length PGRN. R&D MAB2420™ staining revealed punctate labeling after a 24 hour pulse, but the signal was eliminated after the 6 hour chase, indicating that full-length PGRN had been metabolized. These results demonstrate that the R&D AF2420™ antibody, which detects GRNs-1, -2, and -3 (see FIG. 1D), can be used for immunofluorescent labeling of GRNs. In addition, the immunofluorescence data confirm processing of PGRN into stable GRNs and validate a useful tool for studying PGRN/GRN localization inside cells.

Extracellular or newly synthesized PGRN can be routed to the lysosome via multiple mechanisms where it co-localizes with the lysosomal marker, LAMP1. In HeLa cells, significant overlap of LAMP1 and R&D AF2420™ co-staining was observed as has been reported by Chen-Plotkin. See J Soc Neuro, 2012, 32:11213-11227. However, it has been unclear whether full-length PGRN or GRNs are the main species localized to lysosomes. To address this, antibodies were used to detect GRNs and to explore the production and localization of GRNs inside cells. First, density-based gradient centrifugation on HeLa lysates was performed to determine where endogenous PGRN and GRNs fractionate based on organelle-specific proteins (FIG. 5). PGRN was found throughout all of the fractions, which indicates its presence in the endoplasmic reticulum (ER), golgi apparatus, and endosomes. On the other hand, GRNs were exclusively concentrated in fractions that were most enriched for LAMP1, indicating their likely localization to lysosomes. Next, co-localization of GRNs was assessed with organelle markers in PGRN KO cells pulse-chased with PGRN (24 hour pulse/6 hour chase) using double-label immunofluorescence. Again, GRNs (as assessed by R&D AF2420™) showed significant co-localization with the lysosomal marker LAMP1 and did not overlap with markers for early endosomes (Rab5), the golgi apparatus (RCAS1), the ER (calnexin), or mitochondria (COX IV). To analyze the localization of GRNs to lysosomes in more detail, a PGRN pulse-chase assay was coupled with super-resolution microscopy. GRNs were often associated with the inner leaflet of LAMP-1-positive lysosomes suggesting that a portion of GRNs may be associated with the lysosome membrane and/or membrane-bound proteins. Taken together, these experiments indicates that GRNs, rather than PGRN, are the predominant stable species present in lysosomes.

### GRN Levels are Regulated by Expression of SORT1 or TMEM106B

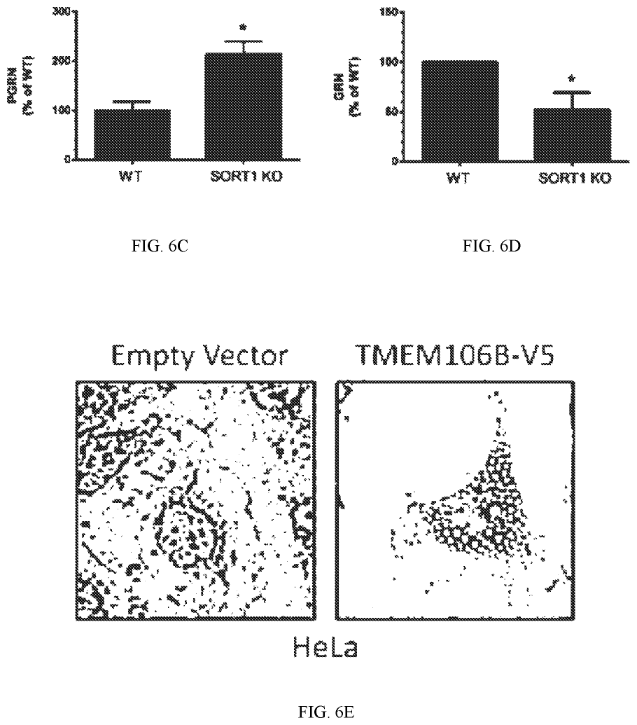

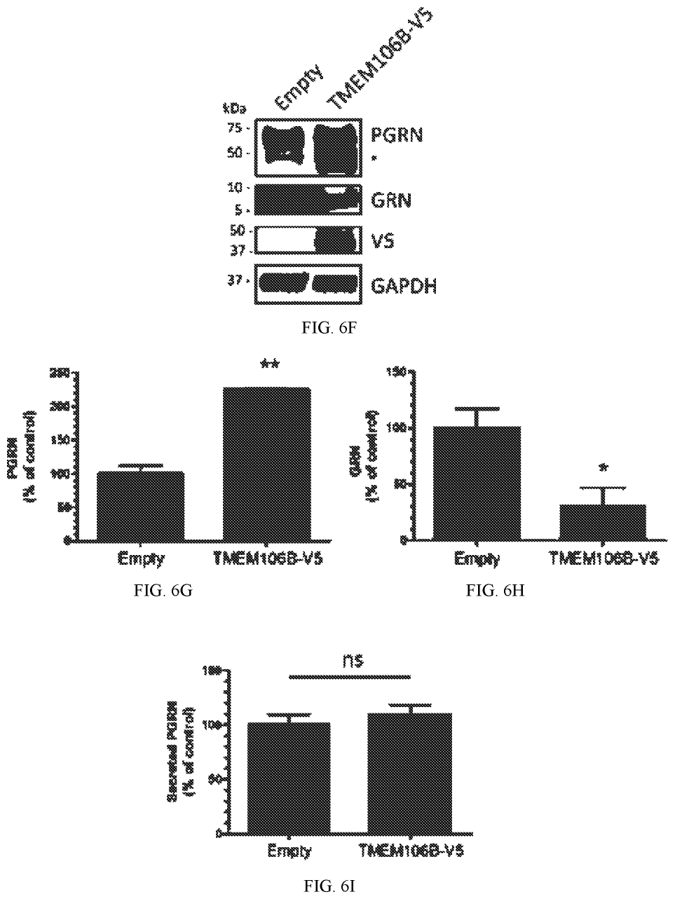

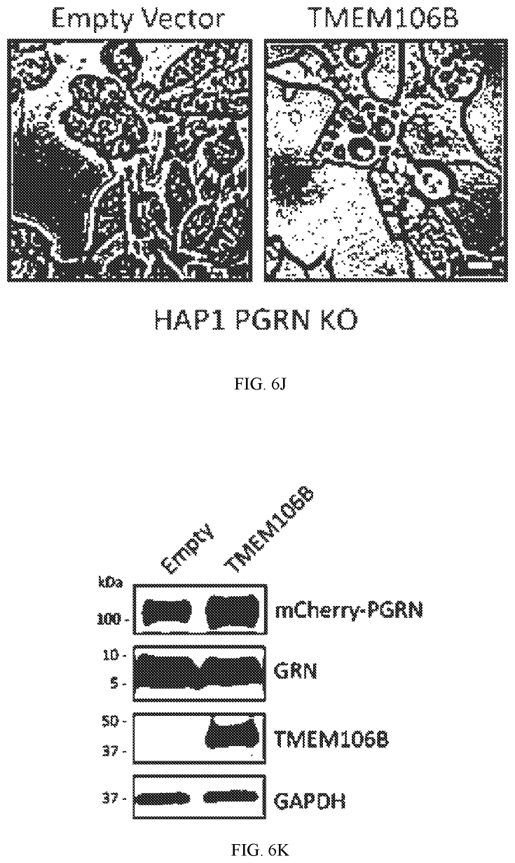

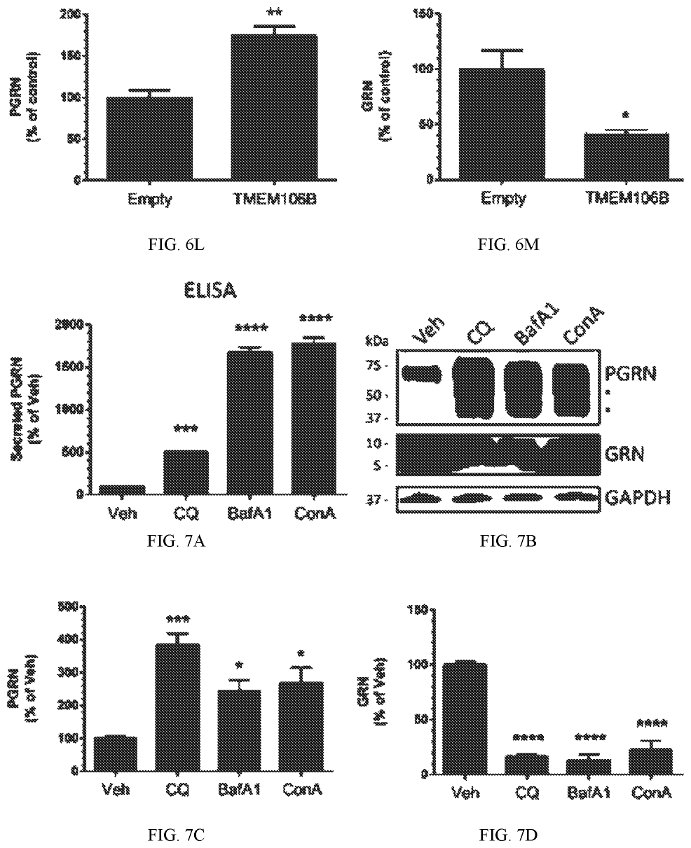

PGRN levels have been linked to expression of several genes including SORT1, encoding the membrane receptor sortilin, and TMEM106B, encoding the transmembrane protein 106B (TMEM106B). Thus, altered expression of these proteins could affect the production of intracellular GRNs as well. To address this, experiments were performed to determine if expression of sortilin, which facilitates endocytosis and trafficking of PGRN to the lysosome, influences GRN levels. PGRN KO cells were treated with PGRN tagged at either the C-terminus (C-TAP), which disrupts sortilin binding or the N-terminus (N-TAP), which preserves sortilin binding, and measured intracellular PGRN and GRN by immunoblot. C-TAP PGRN was not efficiently endocytosed and processed into GRNs compared to N-TAP PGRN (FIG. 6A), indicating that sortilin plays a role in the uptake of extracellular PGRN in these cells. Next, HAP1 SORT1 KO cells were generated using CRISPR/Cas9 technology. It was verified that they do not produce sortilin by immunoblot (FIG. 6B). SORT1 KO cells had significantly increased levels of PGRN (FIG. 6C) and had significantly reduced levels of GRN (FIG. 6D) compared to WT cells. Thus, disruption of the SORT1/PGRN axis alters intracellular PGRN trafficking and leads to decreased GRN production. Moreover, because genetic deletion of SORT1 did not completely eliminate production of GRNs, this data suggests that other pathways exist to traffic PGRN to the lysosome.

Next, experiments were performed to determine if overexpression of the FTD-GRN modifier TMEM106B affects production of GRNs. TMEM106B is a neuronal, lysosomal protein and its overexpression in cells causes lysosomal dysfunction and increased intracellular PGRN levels. Notably, increased levels of TMEM106B have been found in FTD patient brains. Here, TMEM106B was transfected into HeLa cells. PGRN and GRN levels were measured after 48 hours by immunoblot. TMEM106B expression resulted in the accumulation of large vacuoles indicative of impaired lysosomal acidification and function (FIG. 6E) and a significant increase in intracellular PGRN (FIGS. 6F and 6G). Conversely, GRN levels were significantly decreased (FIGS. 6F and 6H). Difference in secreted PGRN levels were not detected with TMEM106B expression (FIG. 6I) indicating that TMEM106B overexpression likely inhibits intracellular processing of PGRN into GRNs. To assess whether TMEM106B overexpression specifically affects processing of endocytosed PGRN, PGRN KO cells were transfected with TMEM106B for 24 hours and then treated them with mCherry™-PGRN for an additional 24 hours. Once again, TMEM106B overexpression in HAP1 cells resulted in enlarged vacuoles (FIG. 6J) and significantly reduced processing of endocytosed PGRN into GRNs (FIGS. 6K, 6L, and 6M). Together, these data indicate that TMEM106B may increase FTD risk by inhibiting the processing of PGRN into GRNs through lysosome dysfunction or altered trafficking of PGRN.

The Processing of PGRN into GRNs is Inhibited by Pharmacologic Lysosome Inhibition and Cysteine Protease Inhibitors

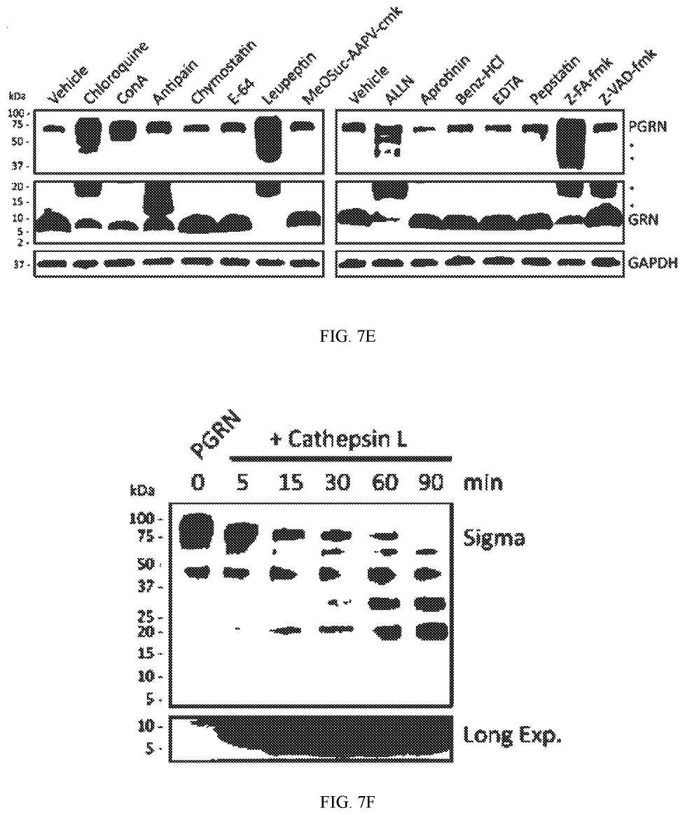

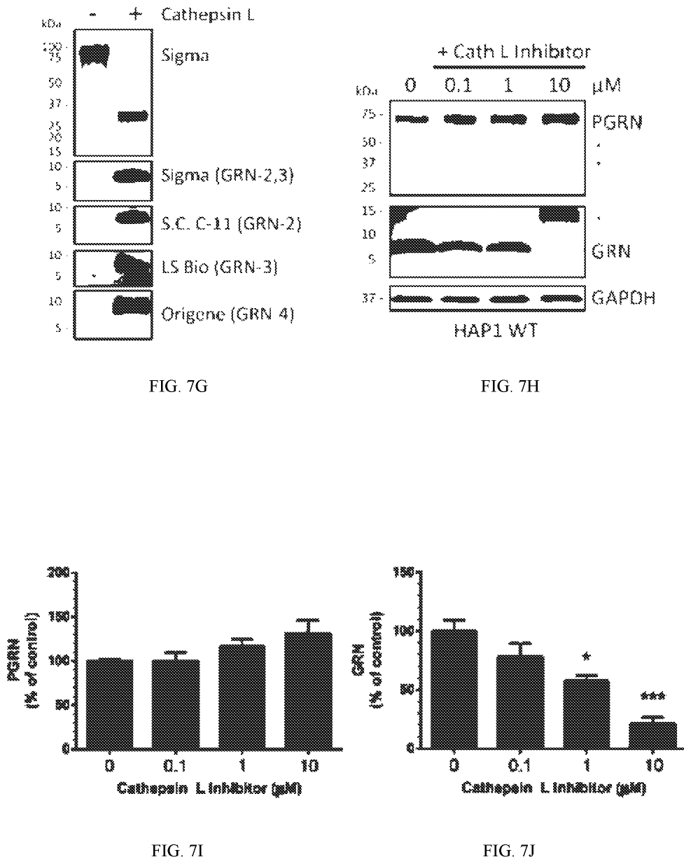

Because rapid processing of endocytosed PGRN into GRNs was observed, inhibiting lysosome function could impair GRN production. To determine the effect of pharmacologic lysosome inhibition on GRN levels, HAP1 WT cells were treated with the pan-lysosomal inhibitors chloroquine, bafilomycin A1, or concanamycin A1 for 24 hours and monitored PGRN/GRN levels by immunoblot. These compounds act as lysosome alkalizing agents or inhibitors of the vacuolar-type H+-ATPase (V-ATPase), which is needed for proper lysosome acidification and function. Further, these compounds increase intracellular and secreted PGRN. In our experiments, both secreted PGRN (FIG. 7A) and intracellular PGRN (FIGS. 7B and 7C) were significantly increased by all of the pan-lysosomal inhibitors. In contrast, GRN levels were significantly decreased (FIGS. 7B and 7D), indicating that the processing of PGRN to GRNs was inhibited and depends on proper lysosome acidification and function.

Processing of PGRN into GRNs likely involves one or more lysosomal proteases. To narrow down which class of proteases may be responsible for intracellular PGRN metabolism, HAP1 WT cells were treated with a panel of protease inhibitors and measured PGRN/GRN levels by immunoblot. Inhibitors of serine, aspartic, metallo-, or trypsin-like proteases did not affect GRN production (FIG. 7E). However, multiple inhibitors of cysteine proteases (antipain, leupeptin, ALLN, and Z-FA-fmk) reduced GRN levels compared to vehicle treated control (FIG. 7E). One potential candidate lysosomal cysteine protease is cathepsin L, which was reported to cleave mouse PGRN in vitro. Incubation of cathepsin L and recombinant human PGRN lead to the time-dependent decrease of full-length PGRN and a corresponding increase of GRN protein (FIG. 7F).

Interestingly, the in vitro cleavage of PGRN by cathepsin L generated multiple, mature GRNs including GRN-2, -3, and -4 (FIG. 7G). Finally, treatment of HAP1 WT cells with the cathepsin L inhibitor II (Z-FY-CHO; Calbiochem™) modestly increased endogenous PGRN (FIGS. 7H and 7I), while significantly decreasing production of GRN (FIGS. 7H and 7J). Overall, these data indicate that properly functioning lysosomes and cysteine proteases in particular contribute to the intracellular processing of PGRN into GRNs.

Human PGRN is Processed into Mature GRNs in Mouse Cells and Brain

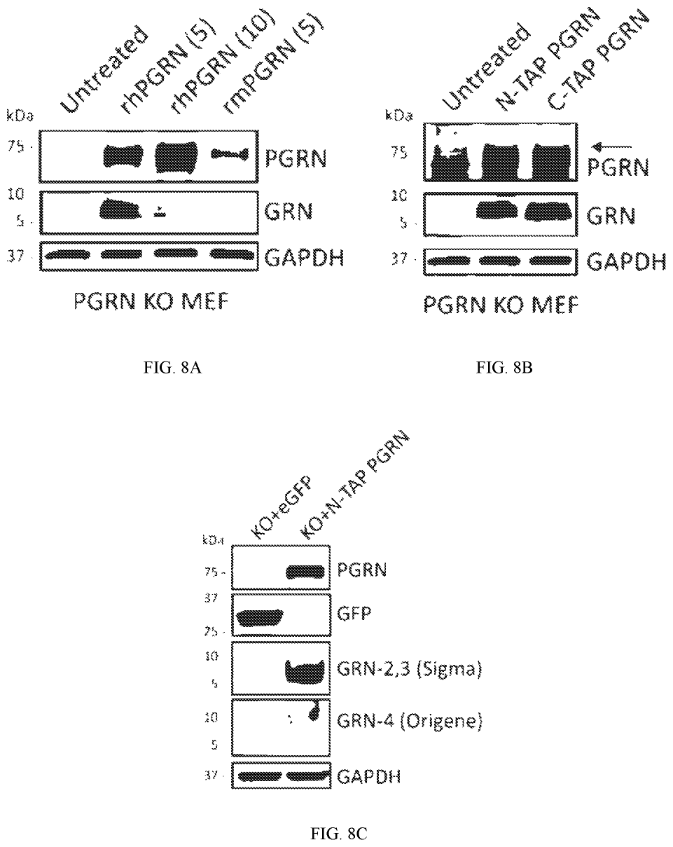

Because this study focused on screening and validating human specific PGRN antibodies, antibodies to detect endogenous mouse GRNs have not been identifies. Nevertheless, to determine if mouse cells are able to process human PGRN into mature GRNs, primary PGRN KO mouse embryonic fibroblasts (MEFs) were treated with recombinant human or mouse PGRN for 48 hours. PGRN/GRN levels were measured in the lysates by immunoblot. PGRN KO MEFs efficiently endocytosed human PGRN and generated mature human GRNs (FIG. 8A). The Sigma™ antibody did not detect any endogenous GRN bands in the PGRN KO MEFs treated with mouse PGRN, confirming that it is specific to human GRN (FIG. 8A). Further, PGRN KO MEFs were able to internalize N-TAP or C-TAP human PGRN and generate GRNs equally well (FIG. 8B) indicating that primary MEFs do not require sortilin for PGRN uptake. Next, experiments were performed to determine if human PGRN would be processed into GRNs in mouse brain. Somatic brain transgenesis (SBT) were performed by injecting rAAV2/1 encoding eGFP or human N-TAP PGRN into the cerebral ventricles of newborn (PO) PGRN KO mice. The SBT paradigm leads to transgene expression that is almost exclusively neuronal as assessed by predominant co-localization between eGFP or PGRN (V5) and the neuronal marker NeuN at 3 months of age. Mice harvested at 14 months of age robustly expressed human PGRN (R&D AF2420™) and produced human GRNs (Sigma™ and Origene™) in brain tissue lysates (FIG. 8C). Taken together, these data demonstrate that the generation of GRNs from PGRN, including the required sorting pathways and protease(s), are conserved between humans and mice.

### Multiple GRNs are Haploinsufficient in FTD-GRN Patient Fibroblasts and Brain

PGRN levels are reduced by approximately 50% in FTD-GRN patient fibroblasts, brain, serum, and CSF, but nothing is known about the relative levels of GRNs in these patients. Using the validated GRN-detecting antibodies from Sigma™ (GRN-2,3) and Origene™ (GRN-4), mature GRN levels were measured in primary human fibroblasts from three FTD-GRN patients compared with three controls (FIG. 9A). Full-length PGRN (FIG. 9 B) as well as GRNs (FIGS. 9C and 9D) were significantly decreased in the FTD-GRN fibroblasts by approximately 50%, compared to controls. GRN levels were assessed in lysates of frontal cortex brain tissue from five FTD-GRN patients compared to five age-matched controls. Once again, multiple GRNs (GRN-2,3 and GRN-4) were significantly reduced by about 60% compared to controls (FIGS. 9E, 9F, and 9G), similar to full-length PGRN, which was reduced by about 40%, as measured by ELISA (FIG. 9H). These data demonstrate that not only PGRN, but also multiple GRNs, are haploinsufficient in FTD-GRN patients and raise the possibility that deficiency of GRNs contributes to the underlying pathogenic process leading to neurodegeneration.

### Administration of Exogenous Recombinant GRN-2

Administration of exogenous recombinant GRN-2 to Grn KO mouse fibroblasts can rescue lysosomal defects, providing compelling evidence that GRNs have a lysosomal function. GRNs (GRN-2 and GRN-4) are haploinsufficient in FTD-GRN patient fibroblasts and brain tissue indicating that deficient GRN levels may initiate lysosome dysfunction leading to neurodegeneration. Exogenous delivery of recombinant GRN-2 to lysosomes can rescue a lysosome defect in Grn KO mouse fibroblasts indicating that a single GRN provides protective function.

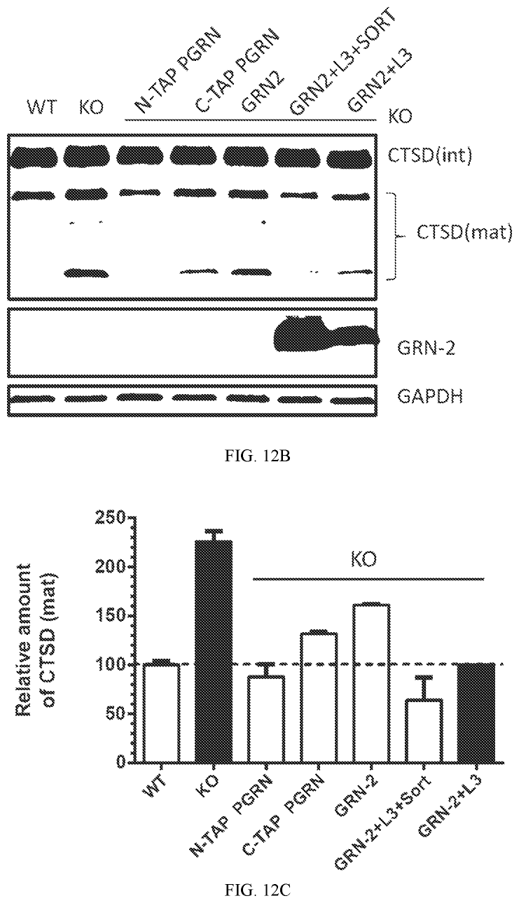

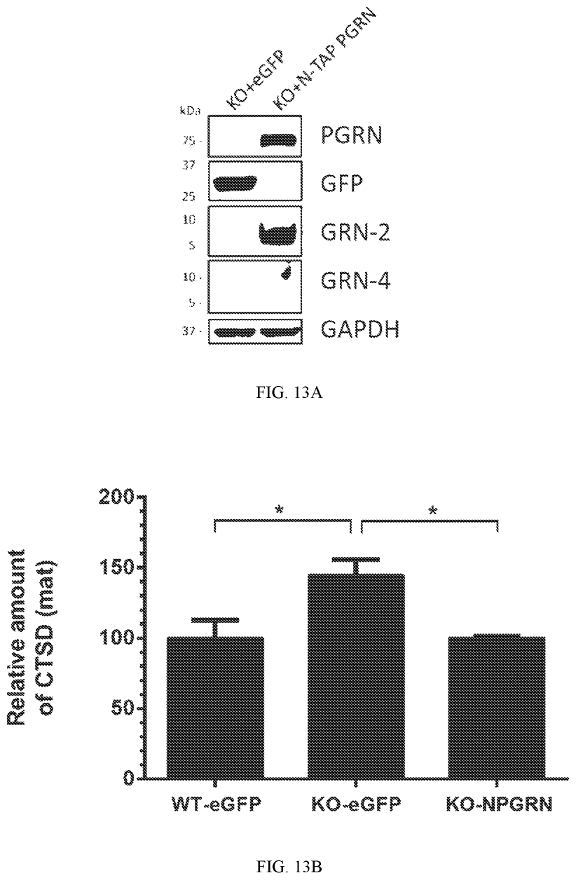

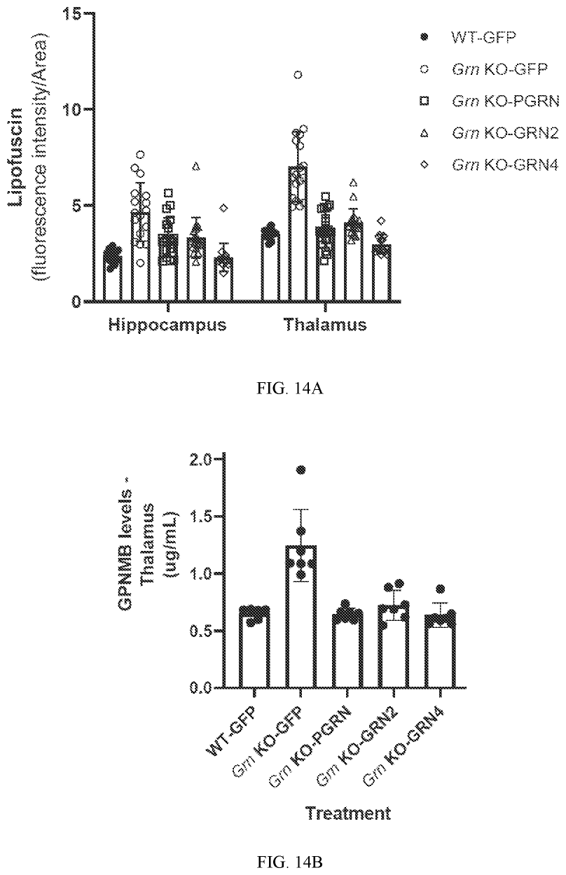

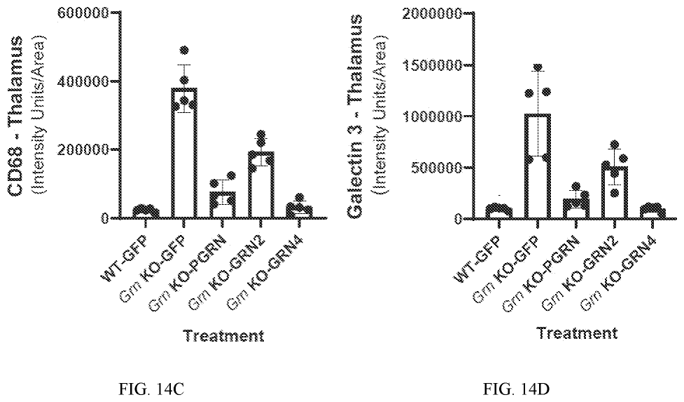

Purified recombinant PGRN were conjugated with a chemical cross-linker (i.e. Sulfo-SBED or SDAD) that labels multiple GRNs directly. PGRN KO cells were treated with labeled PGRN for 24 hours and chase with fresh media for 6 hours. At this timepoint, only lysosomal GRNs, but not PGRN, are detected in cells. Similar experiments can be performed with PGRN KO cells treated with labeled GRN-2+L3, L3 is PTGTHPLAKKLPAQRTNRAVALSS (SEQ ID NO: 34), or GRN-2+L3+SORT, SORT is ALRQLL (SEQ ID NO: 35), which is internalized and routed to lysosomes (FIG. 12A). Grn KO primary mouse embryonic fibroblasts (MEF) can be rescued by exogenous treatment of human PGRN (FIGS. 12B, 12C). Treatment of Grn KO MEFs with exogenous human GRN-2 alone also rescues the CTSD phenotype (FIGS. 12B, 12C). Rescue is dependent on uptake and production of mature GRN-2. These experiments provide evidence that GRNs are important for lysosomal homeostasis and provide rationale for exploring GRN replacement as a therapeutic strategy to treat FTD-GRN.

### Administration of Recombinant Adeno-Associated Virus Encoding PGRN and GRN-2

One can perform ICV injections of recombinant Adeno-Associated Virus encoding human PGRN, GRN-2, or a mCherry™ control into newborn Grn KO mice (PO), which leads to wide-spread transduction of the murine brain. Human PGRN expressed in this manner is processed into mature GRNs in the brain (FIG. 13A), indicating conserved routing and processing pathways. Further, rAAV-hPGRN expression in brain can rescue elevated CTSD levels seen in Grn KO mice (FIG. 13B). It is believed that expression or exogenous replacement of one more lysosomal GRNs in the brain can rescue patho-phenotypic changes seen in PGRN-deficient cell and animal models.

1. A method of treating dementia or cognitive impairment comprising administering an effective amount of a vector encoding a recombinant granulin having the amino acid sequence of SEQ ID NO: 14 (Granulin-2+linker) to a subject in need thereof.

2. The method of claim 1, wherein subject is a human subject.

3. The method of claim 1, wherein administration is by temporal vein injection, intravenous injection, intracerebroventricular injection, or by implantation or exposure into the brain through pressure driven infusion.

4. A method of treating dementia or cognitive impairment comprising administering an effective amount of a vector encoding a recombinant granulin having the amino acid sequence of SEQ ID NO: 16 (Granulin-4+linker) to a subject in need thereof.

5. The method of claim 4, wherein subject is a human subject.

6. The method of claim 4, wherein administration is by temporal vein injection, intravenous injection, intracerebroventricular injection, or by implantation or exposure into the brain through pressure driven infusion.

7. A method of treating dementia or cognitive impairment comprising administering an effective amount of a vector encoding recombinant granulin having the amino acid sequence Z¹CX¹DX²X³TCCX⁴Z²X⁵X⁶X⁷X⁸Z³GCCPMPZ⁴AX⁹CCZ⁵DZ⁶X¹⁰HCCPX¹¹X¹²X¹³X¹⁴CDZ⁷ (SEQ ID NO: 22), wherein each X is, individually and independently, any amino acid and each Z is, individually and independently, a conserved amino acid compared to the corresponding amino acid when aligned with the amino acid sequence of SEQ ID NO: 31, to a subject in need thereof.

8. The method of claim 7, wherein subject is a human subject.

9. The method of claim 7, wherein administration is by temporal vein injection, intravenous injection, intracerebroventricular injection, or by implantation or exposure into the brain through pressure driven infusion.
